(19) United States
(12) Patent Application Publication
(10) Pub. No.: US 2026/0155250 A1
(43) Pub. Date: Jun. 4, 2026

(54) SYSTEM AND PROCESS FOR CHARACTERISING EATING BEHAVIOURS TO USE IN GUIDING THE TREATMENT OF OBESITY

(71) Applicant: Ole SCHMIEDEL, Auckland (NZ)

(72) Inventor: Ole SCHMIEDEL, Auckland (NZ)

(21) Appl. No.: 18/964,550

(22) Filed: Dec. 1, 2024

(30) Foreign Application Priority Data
Oct. 6, 2023 (NZ) 804281

(51) Int. Cl. G16H 50/20; A61B 5/16; G09B 19/00; G16H 10/20; G16H 20/60; G16H 20/70

(52) U.S. Cl. CPC G16H 50/20; G09B 19/0092; G16H 10/20; G16H 20/60; G16H 20/70; A61B 5/16

(57) ABSTRACT

A process of generating data carrying information on a treatment recommendation for a clinician including presenting a set of questions to a subject at a computer implemented user interface, each question having a defined loading of one of a set of behavioural factors, receiving responses to the set of questions from the subject at the computer implemented user interface, quantifying each response to represent a scale of the response, combining the response with a mapping of the item to its respective behavioural factor to generate data carrying information representing the scale of response and mapping of the response to the behavioural factor, combining the quantified responses for each behavioural factor to generate data carrying information on combined responses for each behavioural factor in the set, and applying a computer implemented analytical process to characterise the subject using the behavioural factors to generate data carrying information on a characterisation of the subject by behavioural factor.

### CROSS-REFERENCE TO RELATED APPLICATIONS

This application claims priority to New Zealand Patent Application No. 6741041, filed on 6 Oct. 2023, both of which are incorporated herein by reference in their entireties.

### TECHNICAL FIELD

The present invention relates to systems and methods performed by systems for characterizing subjects by responses to questionnaire items.

### BACKGROUND

Obesity is a chronic relapsing disorder that increases the risk for a variety of medical conditions, such as type 2 diabetes, hypertension, cardiovascular disease and several cancers (Upadhyay, Farr, Perakakis, Ghaly, & Mantzoros, 2018). The global prevalence of obesity continues to rise. Especially concerning are the rising number of people with morbid-obesity, defined by a BMI>40 kg/m2, whose life expectancy is shortened by approximately 8-10 years. Research has shown that losing 5-10% of body weight significantly reduces obesity-related medical complications (Ryan & Yockey, 2017), hence, treating obesity is the most efficacious way to simultaneously treat several obesity-related complications (Apovian et al., 2015). Despite advances in understanding of the pathophysiology of obesity, responses to current weight loss pharmacotherapy are highly variable (Roberts, Christiansen, & Halford, 2017). It has been recognized that there is a need to identify predictors of response to obesity treatment to improve treatment outcomes.

One approach to exploring personalized obesity treatment options is the concept of ‘eating behaviour phenotypes’, which exist in addition to metabolic phenotypes (Roberts et al., 2017). Emerging evidence suggested the existence of eating behaviour phenotypes based on perceived hunger, satiety and satiation and the way nutrition is ingested and metabolized (Acosta et al., 2015; Camilleri, 2019). However, identifying distinctive phenotypes proved difficult. Acosta et al. (Camilleri & Acosta, 2016) use an array of assessments, such as measuring stomach capacity, gastric emptying of solids and liquids, postprandial levels of satiation hormones, and calorie intake in a buffet-style meal, to define different phenotypes. These time and cost-intensive approaches are however mainly suitable for academic settings and alternative methods for identifying actionable eating behaviour traits are of importance in the clinical setting.

In is an object of the present invention to provide a process of indicating treatments likely to have efficacy in treating given subjects or at least to provide the public with a useful choice.

In is an object of the present invention to provide a system capable of indicating eating behavioural traits likely to have efficacy in treating given subjects or at least to provide the public with a useful choice.

### DISCLOSURE OF THE INVENTION

Aspects of the present invention provide a process of identifying an obese subject as being responsive to one of a given set of treatments wherein said process comprises determining eating behaviour characteristics, wherein a characteristic is determined by the subject's response to asset of questionnaire items.

Aspects of the invention allow a subject to be classified as having an eating behaviour trait responsive to an intervention based upon the subject's response to questionnaire items used in the process.

Aspects of the invention allow a subject to be classified as having a defined eating behaviour factor responsive to an intervention based upon the subject's response to questionnaire items used in the process.

Aspects of the invention allow a subject to be classified as having an eating behaviour phenotype responsive to an intervention based upon the response to questionnaire items used in the process.

In another aspect the invention provides a process of generating data carrying information on a treatment recommendation for a clinician, the process comprising the steps of:
presenting a set of questions to a subject at a computer implemented user interface, each question having a defined loading of one of a set of behavioural factors;
receiving responses to the set of questions from the subject at the computer implemented user interface;
quantifying each response to represent a scale of the response;
combining the response with a mapping of the item to its respective behavioural factor to generate data carrying information representing the scale of response and mapping of the response to the behavioural factor;
combining the quantified responses for each behavioural factor to generate data carrying information on combined responses for each behavioural factor in the set;
applying a computer implemented analytical process to characterise the subject using the behavioural factors to generate data carrying information on a characterisation of the subject by behavioural factor.

The process may comprise generating data carrying information which identifies a treatment selected from a set of treatments, the selection made using the characterisation of the subject by behavioural factors.

The process may comprise generating data carrying information which identifies a treatment by identifying a phenotype using the behavioural factor.

The process may generating data carrying information which identifies a treatment using data which associates a behavioural factor with a phenotype to identify a phenotype for the subject.

The process may comprise generating data carrying a selected recommendation from a set of candidate recommendations of a intervention, the recommendation selected using the characterisation.

The process may comprise generating data carrying a selected recommendation using data which associates a set of phenotypes with a set of treatments known to be effective in treating the phenotype.

The process may comprise generating data carrying information which recommends the selected intervention.

The process may comprise:
repeating the steps of the paragraph above in a second iteration of the process of the paragraph above to provide first and second iterations of the process;
generating data carrying information to record a change in the characterised behaviour factors in the subject; and
generating data carrying information on efficacy of the intervention recommended in the first iteration.

The process may comprise storing the data carrying information on responses by scale of response and/or mapping of response to provide updated data to use in selecting behavioural factors.

The process may comprise storing the data carrying information on characterisations to provide updated data to use in selecting behavioural factors.

The behavioural factor may be an eating behavioural factor.

The phenotype may be a behavioural phenotype.

The phenotype may be an eating behavioural phenotype.

Characterising the subject using eating behavioural factors may comprise applying one or more rules to select a behavioural factor for the subject and the recommendation may be selected using the selected behavioural characteristic.

Characterising the subject using eating behavioural factors may comprise applying one or more rules to select a behavioural factor which has the highest combined quantity.

Characterising the subject using eating behavioural factors may comprise applying one or more rules to select a dominant eating behavioural factor.

Combining quantified responses of the subject for the set of questions may comprise finding for each question a product of i) a quantified response to the question and ii) a loading onto an eating behavioural factor defined for each question.

The set of questions presented may be selected from a superset of questions.

Each question may have a known loading of one of a selected set of eating behavioural factors.

The questions may be selected using rules applied to loadings of two or more factors for the same question.

The set of questions are selected so as to have a degree of mutual exclusivity of eating behavioural factors.

An eating behavioural factor may be a metric for how well a question indicates an eating behavioural factor. For example, a loading factor of 1 may indicate that the question perfectly indicates the respective eating behaviour. For example, also a loading factor of 0 may indicate that the question does not indicate the relevant eating behaviour.

The set of questions presented may be selected by applying rules to reduce any cross-loading of eating behavioural factors for each question. For example, the rules applied may deselect questions that significantly indicate two or more eating behaviours.

The set of questions may be selected from a superset of questions that are known to indicate eating behaviours.

The eating behavioural factors may be selected from a superset of eating behavioural factors.

The eating behavioural factors may be selected by exploratory factor analysis.

The eating behavioural factors may be selected as the minimum set of factors that is capable of defining a space that encapsulates a defined percentage of responses of a set of subjects to a superset of questions.

Three eating behavioural factors may be selected from the superset.

Aspects of the present invention provide a process comprising: characterising a subject by eating behaviour phenotype using received responses to a set of questionnaire items the responses received from the subject, wherein each item of the set may be selected to indicate an eating behavioural factor from a defined set of eating behavioural factors.

Aspects of the present invention provide a process comprising: characterising a subject by eating behaviour phenotype using received responses to a set of questionnaire items the responses received from the subject, wherein each item of the set may be selected to indicate an eating behavioural factor from a defined set of eating behavioural factors.

Said indication may be defined as a value of a mapping metric for the item mapping to the eating behavioural factor.

Said describing may be defined as a value of a mapping metric for the item mapping to the eating behavioural factor.

The process may comprise quantifying the received responses.

The process may comprise combining quantified received responses.

The process may comprise characterising the subject by the combined quantified received responses.

Characterising the subject may comprise categorising the subject by eating behavioural factor.

Characterising the subject may comprise applying rules which categorise the subject by a dominant eating behavioural factor.

The process may comprise identifying an eating behavioural trait using the combined quantified received responses.

The process may comprise identifying an eating behavioural phenotype using the combined quantified received responses.

Characterising the subject may comprise applying rules which identify the highest value for a factor for the subject and which reference to two background variables:
(1) the median value (or score) for that eating behaviour (EB) in a whole reference data population (>1SD of the mean); either or
(2) the difference between the individual's dominant vs. second or third eating behaviour score. (>20% difference primary vs next).

This allows the characterisation by eating behaviour to be referenced against two different values, the eating behaviours within one subject, and the strengths of that particular eating behavioural factor in a reference cohort of subjects.

The applicant has observed that this allows the assessment of inter-individual variations in eating behaviour. In one example, each individual will have a specific numerical value between 0 and 100 on each of the three scales. Rules may be applied to characterise the individual, or subject, using the specific numerical value between 0 and 100 on each of the factors. Each said factor may correspond to a different eating behavioural factor. A set of rules to characterise the subject by dominant eating behavioural factor may define that that a subject has a value of their dominant Eating behavioural factor that is greater than one SD [standard deviation] than the mean for this factor. In one example rules to characterise the subject may define that a dominant eating behaviour means that the numerical score of a primary value for a given or the three scales is greater than 20 points (20%) than a closest secondary specific value for a specific values for any other of the three scales.

For example, a person may score 70 in a first scale that corresponds to the primary eating behavioural factor of EE (emotional eating), 45 in another a secondary eating behavioural factor constant hunger (CH) and 30 in a tertiary factor feasting (F). The dominant eating behavioural factor would be a first factor, such as EE (Emotional Eating) for example as it is 25% greater than a second eating behavioural factor CH (Constant Hunger) for example. If the group mean for EE is 50 and 1SD would be +/−15% of the mean (range 35-65), then the rules applied in this example would determine that individual would also have a dominance score in EE. Rules applies may characterise the individual or subject as having a dominant eating behaviour factor of EE.

In an example, the process may apply rules for recommended intervention using the dominant eating behaviour. The rules may select an intervention known to have efficacy for one particular eating behaviour, such, for example emotional eating (EE) and generate data carrying information on a recommendation for this intervention for a clinician treating the subject. It is important here that a recommendation is made to a clinician and the clinician makes all decisions regarding treatment.

The process may comprise adding the responses from the subject to the set of items to a reference data set. This may generate reference data for subjects. This may provide an interactive data repository that constantly acquires new participant data and thereby adapts the combined responses, such as group mean.

The medium value of the factor may be derived from a large group of people who comprise the reference data set. In this example, each factor (EE, CH and F) has a medium value. In one example, the reference data set is the data set established during the tool's different validation stages. In another example, responses to questionnaire items according to any paragraph herein may be used to update the reference data. In one example, responses to questionnaire items according to any paragraph herein may be used to update a data store of reference data.

In some examples, the medium value of the factor is not for an individual but for the reference data set. Each individual will have a unique value in each of the three factors corresponding to the three eating behavioural phenotypes, for examples where the selected eating behavioural phenotypes numbers three. In other examples, there may be four or five selected eating behavioural factors. In these examples, there may be four or five subsets of questionnaire items where items in each subset indicate or describe and map to a different one of the four or five eating behavioural factors. In other examples there may me more selected eating behavioural factors that three, four or five. For example, there may be six or more. Also for example, there may be two.

The eating behavioural factor may be selected from the defined set of eating behavioural factors and using the selected eating behavioural factor to characterise the subject in said characterisation.

The process may comprise selecting the set of questionnaire items using exploratory and confirmatory factorial analysis from the superset of questions by defined loading for behavioural characteristics onto the factors of the three-factor model.

The set of questionnaire items may comprise subsets of questionnaire items, each subset associated with a different eating behavioural factor from the defined set of eating behavioural factors to characterise the subject in said characterisation.

Each questionnaire item may have a behavioural factor mapping value defining how well the questionnaire item indicates and/or describes an eating behavioural factor.

For example, an item with a behavioural factor mapping value of 1 on a scale of 0-1 to an eating behavioural factor may perfectly indicate whether the subject exhibits the eating behavioural factor. This is a mapping factor value of 1 may be a 100% indication. The applicant has observed that there are unlikely to be any known questionnaire items with a behavioural factor mapping value as high as 1.

The process may comprise presenting a set of questionnaire items which each have a sufficient mapping value to one behavioural eating factor of said set of behavioural eating factors.

The set of eating behavioural factors may number three.

The set of eating behavioural factors may relate to eating behaviour phenotypes that relate to physiological processes, such, for example, reduced satiation or fullness at meal times, reduced satiety and increased hunger between meals, and emotional eating.

The set of questionnaire items may comprise subsets of questionnaire items, each subset comprising questionnaire items that best describe the same eating behavioural factor of said set of behavioural eating factors.

The set of questionnaire items may be selected from a superset of candidate questionnaire items having a defined mapping to an eating behavioural factor. The set of questionnaire items may be selected, for example, by the process of factorial analysis, from a superset of candidate questionnaire items having a defined mapping to any eating behavioural factor.

The process may comprise presenting a set of questionnaire items which each have a sufficiently exclusive mapping value to one behavioural eating factor. This may be to the exclusion of other eating behavioural factors. For example, the behavioural eating factor value may be greater than 0.4 for one behavioural eating factor and less than 0.2 for any other behavioural eating factor.

Also for example, the behavioural eating factor value may be greater than 0.4 for one behavioural eating factor and 0.2 less for any other behavioural eating factor.

This may provide that the eating behavioural factors can approximate orthogonality for the selected questionnaire items.

This may provide that the eating behavioural factors can be assumed to be orthogonality for the selected questionnaire items.

The applicant has observed that by selecting items that map to a selected set of eating behavioural factors and which can be assumed to be orthogonal, a questionnaire-based process can indicate one or more eating behavioural factors. The applicant has observed that by assuming eating behavioural factors correlate to eating behaviour phenotypes and/or underlying physiology, a questionnaire-based process can indicate an intervention with known efficacy for the phenotypes and/or underlying physiology.

The questionnaire items may be selected from a known superset of questionnaire items known to indicate eating behaviour factors.

The questionnaire items may be selected from the superset by a process of factor analysis.

The questionnaire items may be selected from the superset by so that each selected questionnaire item maps sufficiently exclusively to a definable to one eating behavioural factor so that the factors can be assumed to be orthogonal.

The questionnaire items may be selected from the superset by so that each selected questionnaire item maps sufficiently exclusively to a definable to one eating behavioural factor so that the factors can be assumed to be independent of each other. A set of factors that the selected questionnaire items map reasonably exclusively to will thereby allow the set of factors to provide a mathematical basis or set of coordinates with which to characterise a subject.

The applicant has observed that a superset of known questionnaire items that has some known mapping to a superset of known eating behavioural factors will allow selection of items that map each map approximately exclusively to one eating behavioural factor. The applicant has observed that this allows the set eating behavioural factors to be treated as approximately independent characteristics that can be used to characterise a subject. The applicant has further observed that a set of minimalistic set of eating behavioural factors can be selected by factor analysis. For example, a subject that has responded to a set of questions with high quantitative responses to items that map to one particular factor may indicate that factor as dominant. The subject may then be characterised with reference to that eating behavioural factor, such as having a that factor as their dominant eating behavioural factor. The applicant has further observed that an assumption that eating behavioural factors correlate to underlying physiology and/or phenotypes allows a questionnaire-based process to identifying actionable eating behaviour phenotype that may help in the selection of targeted interventions based on those eating behaviour phenotypes.

The process may comprise finding a product of a mapping value for an item to a behavioural factor and a quantitative response to the questionnaire item input by the subject. Here, ‘product’ may be found by scalar multiplication.

The process may combine said products for the questions in a subset of items associated with the same eating behavioural factor.

The process may comprise combining said products for each eating behavioural factor. This may be so as to determine a combined value for each eating behavioural factor.

Said combining may comprise finding a mean.

Said combining may comprise finding a median.

Said combining may comprise finding a total.

The process may comprise applying rules to the combined values for the set of eating behavioural factors to characterise the subject by eating behavioural factor.

The rules applied to the combined values for the set of eating behavioural factors may select an eating behavioural factor. The process may comprise generating data to identify the subject as characterised by the selected eating behavioural factor.

The rules applied to the combined values for the set of eating behavioural factors may select a dominant eating behavioural factor. The process may comprise generating data to identify the subject as characterised by the identified dominant eating behavioural factor.

The rules applied to the combined values for the set of eating behavioural factors may determine that no eating behavioural factor is sufficiently dominant. The process may comprise indicating that the subject cannot be characterised in the event that the rules applied to the combined values for the set of eating behavioural factors determine that no eating behavioural factor is sufficiently dominant.

The set of questionnaire items may be selected from a superset of candidate items by applying rules to select items that each predict one eating behavioural factor to the exclusion of other eating behavioural factors. In one example the rules may deselect items which have significant mapping to two eating behavioural factors. The rules may attempt to select a set of items which each approximately map exclusively to one eating behavioural factor to the exclusion of other eating behaviour factors.

The process may comprise displaying rules to a user the rules defining how the subject provides responses. This may allow the process to be run with a subject without supervision.

The process may comprise repeating the process of any paragraph above on the same subject after a medical treatment and recording changes that can be observed in eating behaviour after the treatment internation. This may identify changes in eating behaviours after an intervention.

The process may comprise generating data carrying information which identifies a pharmacotherapy, nutritional intervention, or surgical treatment.

The process may comprise generating data carrying information which indicates predicts responsiveness of the subject to a particular treatment that a clinician may consider.

This may allow the process to predict responsiveness to a given intervention by using the individual's responses to the questions. The process may allow the individual to choose intervention A over intervention B, predicting to achieve better outcomes in the desired measures (weight loss) compared with random choice of the intervention.

Classifying said human as having an eating behaviour phenotype responsive to an intervention based upon the response to all items in said tool.

The process may comprise classifying the subject as having an eating behaviour factor and indicating an eating behaviour phenotype that is known to be responsive to a known intervention.

The process may comprise:
applying rules using the eating behavioural factor to select a recommendation of a medical intervention from a set of recommendations of medical interventions; and
generating data carrying information on the selected recommendation.

The process may comprise:
applying rules using the dominant eating behavioural factor to select a recommendation of a medical intervention from a set of recommendations of medical interventions; and
generating data carrying information on the selected recommendation.

The process may comprise:
applying rules using the combined values to select a phenotype known to be response to a medical intervention from a set of phenotypes; and
generating data carrying information on the phenotype.

The process may comprise:
applying rules using the combined values to select a phenotype known to be response to a medical intervention from a set of phenotypes; and
generating data carrying information on the intervention.

Characterising the subject by a dominant eating behaviour from received quantitative responses to a set of items may comprise combining said product for each behavioural factor over the set of items.

The set of eating behavioural factors may be selected from a superset of known eating behavioural factors.

In one particular embodiments, a set of eating behavioural factors may be selected from a superset of eating behavioural factors so as to be a minimal set of factors that define a space which encapsulates a defined portion of responses to a sample set of responses from a sample of subjects to a superset of items.

In another embodiment factor, in the exploratory factor analysis is created by a set of items or questions that cluster together by describing a similar observation,

Characterising the subject may comprise combining quantitative responses from the subject to the set of items that indicates each eating behavioural factor. For example, responses for all items may be combined to provide a combined quantity for each behavioural factor for each subject.

Rules to identify a dominant eating behaviour may be applied to said combined quantities for each subject.

Rules to identify a dominant eating behaviour may be applied to said combined quantity for each subject.

In one particular embodiment, characterising the subject may comprise finding a product of a defined mapping of an item to an eating behavioural factor and a quantitative response received from the subject to an item.

The items may be selected from a set of items which have a known mapping to each eating behaviour factor. Mapping may describe the ability of an item to predict an eating behaviour. For example, a mapping of 1 on a scale of 0 to 1 may indicate that a quantitative response of 1 on a scale of o to 1 will predict the eating behaviour as present in a subject.

A questionnaire item may comprise a question.

A questionnaire item may comprise an image.

In one example a set of questions is presented to a subject and quantitative responses to each question is received. This may be a quantitative value of 1 for strongly agree, for example, and a quantitative value of 0 for strongly disagree.

The eating behavioural factors may be created by factor analysis on a sample of responses to a superset of questionnaire items where the responses are provided by a sample of subjects.

The factor analysis on a sample of responses to a superset of questionnaire items may comprise exploratory factor analysis to determine the dimensionality.

The exploratory factor analysis may be performed using a subset of the sample of responses.

The exploratory factor analysis may comprise applying factor-retaining rules which retain a selection of factors represented in the superset of questionnaire items which have an eigenvalue that meets a defined criteria. The defined criteria for eigenvalues may be greater than or equal to approximately 1. The defined criteria for eigenvalues may be greater than or equal to 1.

The exploratory factor analysis may comprise applying factor-retaining analysis which retain a selection of factors represented in the superset of questionnaire items which account for a defined proportion of variance in the sample responses. The defined proportion may be 85%. factor-retaining analysis may be factor-retaining rules.

The exploratory factor analysis may comprise retaining a selection of factors represented in the superset of questionnaire items which account for a sufficiently high proportion of variance in the sample responses.

The questionnaire items may be selected by defined factor loading value.

The questionnaire items may be selected by applying item-selection rules which cause stepwise elimination of items with insufficient factor loading value.

The questionnaire items may be selected by applying item-selection rules which cause stepwise elimination of items with insufficient factor loading value with the exception of questionnaire items which have a principal loading factor value of greater than or equal to 0.4 a secondary loading factor value of greater than or equal to approximately 0.3 and a difference in loading factor values of greater than or equal to approximately 0.2.

The questionnaire items may be selected by applying item-selection rules to rules which cause stepwise elimination of items with factor loading value below a defined cut-off. The defined cut-off may be approximately 0.4. The defined cut-off may be 0.4.

The questionnaire items may be selected by applying item-selection rules which cause stepwise elimination of items with cross-loading beginning with a uniqueness of greater than or equal to approximately 0.5.

The questionnaire items may be selected by applying item-selection rules which cause stepwise elimination of items with factor loading value of two or more factors.

The questionnaire items may be selected by applying item-selection rules which cause stepwise elimination of items with factor loading value of two or more factors.

The questionnaire items may be selected by applying item-selection rules which cause stepwise elimination of items with factor cross-loading.

The factor analysis on a sample of responses to a superset of questionnaire items may comprise confirmatory factor analysis on a second subset of the sample of responses.

Characterising the subject may comprise classifying the subject into one of the selected eating behavioural factors.

Said classifying may comprise assigning the subject to a category based on the combined product of response and loading factor among the selected eating behavioural factors.

Said classifying may comprise assigning the subject to a category based on the highest median of combined product of response and loading factor among the selected eating behavioural factors.

The process may comprise characterising the subject using combined values for two or more of the selected eating behavioural factors.

Aspects of the present invention provide a process of providing information for use in treatment of obesity in a human, wherein said process comprises:
administering a test operative to diagnose eating behaviours in a said human;
identifying an intervention based on said eating behaviour characteristic obtained from said human by administering said test operative to diagnose eating behaviours.

Aspects of the present invention provide a process of providing information for use in treatment of obesity in a human, wherein said process comprises:
administering a test operative to identify eating behaviours traits in a said human;
identifying an intervention based on said eating behaviour characteristic obtained from said human by administering said test operative to diagnose eating behaviours.

Aspects of the present invention provide a process of providing information for use in treatment of obesity in a human, wherein said process comprises:
administering a test operative to identify eating behaviour phenotypes in a said human;
identifying an intervention based on said eating behaviour characteristic obtained from said human by administering said test operative to diagnose eating behaviours;
instructing a human regarding rules for said test; and administering an intervention to said human.

The human may be a subject of the administered test.

The administered test may comprise characterising using received responses of the subject to a set of questionnaire items the responses, wherein each item of the set may be selected to indicate an eating behavioural factor from a defined set of eating behavioural factors.

The indicated set of eating behaviour factors may indicate a diagnosis.

Said indication of eating behaviour factors may be defined as a value of a mapping metric for the item mapping to the eating behavioural factor.

Said describing may be defined as a value of a mapping metric for the item mapping to the eating behavioural factor.

The process may comprise quantifying the received responses.

The process may comprise categorising the subject by eating behavioural factor.

The process may comprise applying rules which categorise the subject by a dominant eating behavioural factor.

The process may comprise applying rules which identify the highest value for a factor for the subject and which reference to two background variables:
(1) the median value (or score) for that eating behaviour (EB) in a whole reference data population (>1SD of the mean); or
(2) the difference between the individual's dominant vs. second or third eating behaviour score. (>20% difference primary vs next).

This mapping allows the characterisation by eating behaviour to be referenced against two different values, the eating behaviours within one subject, and the strength of that particular eating behavioural factor in a reference cohort of subjects.

Said test is administered to a said human.

Said instructing may comprise providing rules for performing said test.

The process of any paragraph above may comprise said administering said test utilizing a computerised system and analytics algorithm.

The process of any paragraph above wherein said administering comprises recording responses to test questions.

The process of any paragraph above wherein said administering further comprises recording data related to said responses.

The process of any paragraph above may further comprise analysing said responses and said data relative to previously recorded data sets.

The process of any paragraph above wherein said previously recorded data records are obtained during previous administration of the said test The process of any paragraph above wherein said previously recorded data records are normative data for a population previously administered said test.

The process of any paragraph above wherein said previously recorded data records are normative data for a sample of a population, the sample previously administered said test.

The process of any paragraph above may further comprise transmitting said data to a remote device.

The process of any paragraph above wherein said administering may comprise providing a plurality of tests questions administered in sequence.

The process of any paragraph above wherein said analysing comprises characterising said human by identified eating behaviour. Said eating behaviour may be a trait. Said eating behaviour may be a defied factor. Said factor may be identified by factor analysis using a data for responses to a superset of questionnaire items from a population of subjects. Said factor may be identified by factor analysis using a data for responses to a superset of questionnaire items from a sample of a population of subjects.

The process of any paragraph above wherein said analysing comprises identifying eating behaviour traits of said human or said subject.

The process of any paragraph above wherein said analysing comprises identifying eating behaviour characteristics of said human or said subject.

The process of any paragraph above may further comprise selectively repeating said administering for a plurality of discrete tests.

Aspects of the present invention provide a computer implemented tool comprising:
a testing module operative to administer a test to a subject at a computer-generated user interface; and
an instruction module operative to generate instructions at the computer-generated user interface for a subject regarding rules for said test.

Said test may comprise the process of any paragraph herein.

The system of any paragraph above wherein said testing module and said instruction module are implemented in computer software and an analytics algorithm.

The system of any paragraph above further comprising a structure operative to record and analyse responses to the test.

The system of any paragraph above further comprising an analytic module operative to analyse said responses and said data relative to previously recorded data recordings.

Aspects of the present invention provide a computer implemented system comprising:
a testing module operative to administer a test; and
an instruction module operative to instruct a subject regarding rules for said test.
said test may comprise the process of any paragraph herein.

The system of any paragraph above wherein said testing module and said instruction module are implemented in computer software and an analytics algorithm.

The system of any paragraph above further comprising a structure operative to record and analyse responses to the test.

The system of any paragraph above further comprising an analytic module operative to analyse said responses and said data relative to previously recorded data recordings.

The process of administering a sequence of tests comprising of:
a plurality of test questions operative to diagnose eating behaviour characteristics of said human;
instructing a subject regarding rules for responding to said test, administering said test; recording responses to tests; and
administering the test without direct support of a professional.

The process or any paragraph above wherein said instructing, administering, recording, and measuring comprise utilizing a computerized system.

The system of any paragraph above wherein said testing module, instruction module, and test analysis are conducted in computer software.

The process of selecting a treatment regimen for treating said human using said process comprising: selecting a test operative to evaluate eating behaviour, instructing a subject regarding rules for said test, administering said test, recording responses to said test, selecting treatment in accordance with the responses to said test. Selecting a treatment may comprise generating data indicating a treatment.

A process of any paragraph above whereby selectively repeating said test, by said instructing, said administering, said recording, and said measuring, said evaluating and selecting a treatment regimen using a comparison of results obtained during previous administration of said test to said individual.

A process of any paragraph above wherein said treatment regimens comprising administering a pharmacotherapy, a nutritional intervention, a surgical intervention to said human.

Aspects of the present invention provide a process for identifying an obese mammal as being responsive to said treatment with an intervention, wherein said process comprises:
determining an eating behaviour phenotype with said methods in said human, wherein said phenotype is determined by the response of said human to all items in said tool; and
classifying said human as having an eating behaviour phenotype responsive to an intervention based upon the responses to all items in said tool.

The tool may be a questionnaire-based assessment tool.

An item may be a questionnaire item.

An item may be presented as a statement or a question.

The tool may be adapted to allow the participant to indicate their level of agreement with each statement or question.

The tool may be adapted to allow the participant to indicate their level of agreement with each questionnaire item.

The tool may entails different item presentations.

Each item may have a corresponding description of the anchor points.

The tool may be adapted to allow the participant to indicate the level of their subjective sensations.

The tool may be adapted to allow the participant to respond with a scales to questionnaire items.

The scale may be 0-100.

The tool may be adapted to allow the participant to respond with a scales to questionnaire items.

The tool may be adapted to allow the participant to respond by using visual analogue scales (VAS) for all questions.

Aspects of the present invention provide a process for identifying an obese mammal as being responsive to said treatment with an intervention, wherein said process comprises:
determining an eating behaviour trait with said methods in said human, wherein said phenotype is determined by the response of said human to all items in said tool; and
classifying said human as having an eating behaviour phenotype responsive to an intervention based upon the responses to all items in said tool.

An item may be a questionnaire item.

An item may be presented as a statement or a question.

The tool may be adapted to allow the participant to indicate their level of agreement with each statement or question.

The tool may be adapted to allow the participant to indicate their level of agreement with each questionnaire item.

The tool may entails different item presentations.

Each item may have a corresponding description of the anchor points.

The tool may be adapted to allow the participant to indicate the level of their subjective sensations.

The tool may be adapted to allow the participant to respond with a scales to questionnaire items.

The scale may be 0-100.

The tool may be adapted to allow the participant to respond with a scales to questionnaire items.

The eating behaviour phenotype may be determined by a response pattern to the set of questionnaire items presented.

The eating behaviour phenotype may be one of a set of defined four main eating behaviour phenotypes.

The eating behaviour trait may be determined by a response pattern to the set of questionnaire items presented.

The eating behaviour trait may be one of a set of defined eating behaviour traits.

A distinct response pattern to the questionnaire can be identified as present in each of the four main eating behaviour phenotypes.

The tool may be adapted to allow the participant to respond by using visual analogue scales (VAS) for all questions.

Aspects of the present invention provide a process for identifying an obese mammal as being responsive to said treatment with an intervention, wherein said process comprises:
indicating an eating behaviour trait in said mammal, wherein said trait is determined by the response of said mammal to a set of items presented; and
classifying said mammal as having a defined eating behaviour trait based upon the responses to the set of items presented, wherein the trait is known to be responsive to said intervention.

Aspects of the present invention provide a process for identifying an obese mammal as being responsive to said treatment with an intervention, wherein said process comprises:
indicating an eating behaviour phenotype with said methods in said human, wherein said trait is determined by the response of said mammal to a set of items presented; and
classifying said mammal as having an eating behaviour phenotype based upon the responses to the set of items presented, wherein the phenotype is known to be responsive to an intervention.

Aspects of the present invention provide a process for identifying an obese mammal as being responsive to said treatment with an intervention, wherein said process comprises:
indicating an eating behaviour phenotype with said methods in said human, wherein said phenotype is determined by the response of said human to all items in said tool; and
classifying said human as having an eating behaviour trait responsive to an intervention based upon the responses to all items in said tool.

Aspects of the present invention provide a process for identifying an obese subject as being responsive to said treatment with an intervention, wherein said process comprises:
indicating an eating behaviour phenotype with said methods in said human, wherein said phenotype is determined by the response of said human to a set of items presented to the mammal; and classifying said subject as having an eating behaviour trait responsive to an intervention based upon the responses to all items in said tool.

Aspects of the present invention provide a process for identifying an obese mammal as being responsive to said treatment with an intervention, wherein said process comprises:
indicating an eating behaviour phenotype with said methods in said human, wherein said phenotype is determined by the response of said human to all items in said tool; and
classifying said human as having an eating behaviour phenotype responsive to an intervention based upon the responses to all items in said tool.

Said items may be questionnaire items as defined in any paragraph herein.

A process of any paragraph herein wherein said response to the methods described herein comprises a presence of low satiety phenotype, and an absence of low satiation and absence of emotional eating phenotype; and wherein said human is responsive to intervention with an appetite suppressant, for example phentermine pharmacotherapy or other appetite suppressants.

A process of claim any paragraph above, wherein said response to the methods described herein comprises a presence of low satiation phenotype, and an absence of low satiety and absence of emotional eating phenotype; and wherein said human is responsive to intervention with a glucagon like peptide agonist pharmacotherapy, for example group consisting of liraglutide or semaglutide or dulaglutide.

Alternative aspects of the invention to any of the aspects or embodiments of any paragraph herein may substitute rules applied to the combined values for the set of eating behavioural factors with any known alternative, such as machine learning, to characterise the subject by behavioural factor(s).

A process of claim any paragraph above wherein said response to the methods described herein comprises a presence of emotional eating phenotype, and an absence of low satiation and absence of low satiation phenotype; and wherein said human is responsive to intervention with, for example a combination of naltrexone and bupropion or an antiepileptic such as topiramate.

A process of any paragraph above wherein said intervention based on said phenotype is effective to reduce total body weight of said human between 3-15 kg compared to an individual who did not receive treatment

A process of any paragraph above wherein said intervention is effective to reduce waist circumference of said human between 5-15 cm compared to an individual who did not receive treatment

A process of any paragraph above wherein treating an individual with obesity with said treatments based on said methods also treats obesity associated conditions such as diabetes, hypertension (high blood pressure), metabolic associated fatty liver disease, polycystic ovarian syndrome and other related obesity associated diseases.

The methods described herein may also be used additional purposes, for example for the selection of patients for bariatric surgery, to improve response the procedure, or said methods may be used to predict long term success after bariatric surgery.

In further embodiments there is a system for treating a human or mammal subject comprising:
a data store with data carrying information to present as a set of questionnaire items to a subject;
a data store with data carrying information defining a mapping of each questionnaire item to a respective behaviour factor of a behavioural factor model having a set of behaviour factors;
a testing processor operable generate data for a user interface to present the set of questionnaire items to a subject and receive control inputs from the subject and generate data carrying information quantifying each response;
an analysing processor operable to perform computer analysis to identify a behavioural factor for the subject from responses to questionnaire items combined with mapping data; and
a data store with data associating behavioural factors to treatments.

The treatments may be known to be effective for phenotypes which correlate with the behavioural factors.

The analysing tool may use a model having multiple behavioural factors.

The multiple behavioural factors may be selected dependent on a set of data from responses of a sample of subjects. The multiple behavioural factors may be selected dependent on a set of data from responses of a sample of subjects combined with mappings of questionnaire items with respective factors. The factors may be selected dependent on the variance of the subjects responses to a set of questionnaire items.

An eating behavioural factor may be an eating behavioural trait.

A loading onto a factor may be a mapping onto a factor.

As used herein the term ‘characterise’ is intended to generate a description using defined characteristics and includes a description using defined characteristics and metrics associated with those characteristics and includes a description of being assigned to a defined category. For example, a subject may be characterized as having a dominant defined trait and/or behaviour. In another example, a subject may be characterized by one or more quantitative values assigned to defined characteristics.

### BRIEF DESCRIPTION OF THE DRAWINGS

Further and additional aspects of the present invention may be apparent from the illustrations of embodiments of the invention with reference to the drawings in which:

FIG. 1 is a simplified block diagram illustrating the different components of one embodiment of the testing apparatus.

FIG. 2 is a simplified flow diagram illustrating the general operation of one embodiment of a process of instructing and preparing a test subject, facilitating administration and test reliability.

FIG. 3 is a simplified flow diagram illustrating the general operation of one embodiment of a process of performing the test.

FIG. 4 is a simplified flow diagram illustrating the general operation of one embodiment of a process of ascertaining the efficacy of a treatment regime.

FIG. 5 is a line graph depicting the Eigenvalues and Factor Numbers of the Initial Exploratory Factor Analysis Model.

FIG. 6 is a simplified diagram showing the Three-Factor Model of Eating Behaviour Type classification of the Confirmatory Factor Analysis (CFA), depicting *Standardized Factor Loadings and **R² values.

FIG. 7 depicts the associations between total scores (0-80) on the assessment tool and Body Mass Index (kg/m²).

FIG. 8 (a-d) depicts the associations between the three eating behaviour specific scores (0-80) on the assessment tool and Body Mass Index (kg/m²).

FIG. 9 is a simplified flow diagram illustrating exemplary pharmacological treatment interventions for the eating behaviour groups identified, at least in part, based on eating behaviour phenotypes.

FIG. 10 shows a process of generating data with information on recommended treatments for a clinician to consider according to a further embodiment of the present invention;

FIG. 11 shows a process of quantifying a response from a subject to a questionnaire item according to the embodiment of the invention of FIG. 10.

FIG. 12 shows a process of combining quantified responses according to the embodiment of the invention of FIGS. 10 and 11.

FIG. 13 shows a set of quantified responses to questionnaire items in an embodiment of the resent invention.

### DETAILED DESCRIPTION

This description contains methods and systems for use in assessing and treating human obesity. In some circumstances, this document includes methods and systems for determining whether an obese person is responsive to a pharmaceutical intervention. The results of the methods described herein, for example, can determine whether an obese human is likely to respond to an intervention (e.g., pharmacological intervention, surgical intervention, weight loss device, diet intervention, behaviour intervention) based, at least in part, on the assessment tool results.

For example, the methods and systems described herein are based on the concept of distinguishing eating behaviour traits by applying a questionnaire-based assessment tool. Satiety, satiation and emotional eating are potential actionable eating behaviour characteristics of an individual (Acosta et al., 2015; Acosta et al., 2021) that can be targeted by administering one or more interventions (e.g., pharmacological intervention, surgical intervention, weight loss device, diet intervention, behaviour intervention). Potential items for inclusion were selected from prior art, modified and adjusted for the novel assessment tool, and several items were developed de novo by the applicant based on underlying concepts. Items included those that describing emotional eating, meal initiation due to declining satiety after a previous meal, and items that describe meal termination. For example, questions about eating speed, binge eating, food cravings, and restraint, were excluded in the iterative factor analysis during the development phase of the tool as these did not align with the three-factor model. The reader will recognise using a three-factor model as selecting a set of factors from a superset of factors.

Distinguishing eating behaviour traits and/or using eating behaviour characteristics in reference to an individual may be referred herein as characterising the individual. The individual may be referred to herein as a subject in reference to a process in which the individual responds to questions or questionnaire items.

All items were adapted by changing how the item is presented, either as a statement or a question, and how the participant can respond by using visual analogue scales (VAS) for all questions. As described herein, a consistent question style in the form of visual analogue scale (VAS) was used to allow factor analysis and identification of items that best describe the particular eating behaviour style. For example, validated visual analogue scale (VAS) was thought to be preferable as it allows participants to indicate the level of their subjective sensations, giving them a range of possibilities instead of discrete categories, and allowing them to identify small differences in the measured item. VAS which are used to describe sensations on a continuous, unidimensional scale of 0-100 with discrete anchor points on either end with verbal description representing the extreme points of the item measured. For example, such scales measure the participants' level of agreement with each statement or question. They are highly sensitive measurement tools capturing changes in the measured item, making them useful for detecting subtle variations in subjective experiences over time or in response to interventions. For example, the tool described herein entails different item presentations, and each item has its corresponding description of the anchor points.

These items may be referred to herein as questionnaire items.

In some cases, the methods and systems described herein utilize question style items with a unique corresponding response format. For example, “When you are going through a stressful or upsetting setting time, what happens to your eating?” the choices are: anchor 0=I go off food completely, mid-scale 50=undecided, anchor 100=I find it difficult to control my eating and eat much more. “Do you eat large portion sizes?” The choices are anchor 0=definitely false, mid-scale 50=undecided, and anchor 100=definitely true. For example, the questions were randomly positioned in the final tool, avoiding similar questions in close proximity. For example, the assessment process described herein also entails image based questions to improve answer precision and reduce questionnaire fatigue. Image based questions are generally more engaging, simple to answer, and can achieve sufficient quality of responses. The image-based questions were designed based on the concept of vividness of imagery and mental imagery affecting food cravings and provide images with familiarity, arousal and varying in valence (Seo, Rockers, & Kim, 2023).

In some cases, the methods and systems described herein use additional attention checker items to ensure better participant engagement to assess every single item with persistent attention throughout the process of completing the assessment. Attention checker items, for example, include decoy questions, validation, or verification questions. In examples, the methods and systems described herein use attention checker items that allow a random selection of similar items with no preference between them, and images that encourage participants to describe their thoughts and emotions in verbal form to achieve sustained attention. Examples of questions in phenotype group 1 include, without limitation, questions that assess eating initiated in response to the sensation of hunger, the desire eat frequently, strong cravings for food most of the time, eating in the evening before going to bed and hunger between meals. For example, in phenotype group 1 there is absence of questions that describe eating in response to the sensation of negative emotions or the inability to stop eating when having access to unlimited amount of food.

Examples of questions in phenotype group 2 include, without limitation, questions that assess the reduced sensation of fullness during a meal, the likelihood to overeat at a meal, having large portion sizes and rapid return to hunger after a previous meal. For example, in phenotype group 2 there is absence of questions that describe eating in response to the sensation of negative emotions or questions that describe eating in response to the sensation of hunger.

Examples of questions in phenotype group 3 include, without limitation, questions that assess eating in response to a variety of negative emotions. Such negative emotions can be anxiety, feeling tense or worried, or being in a state of depression and having low mood. Examples of negative emotions furthermore include situations when an individual is confronted with a stressful, tense or worrisome situation, and items that eating in the context of negative social interactions or isolation. For example, in phenotype group 3 there is absence of questions that describe eating in response to the sensation of hunger or the inability to stop eating when having access to unlimited amount of food.

As described herein, a distinct response pattern to the questionnaire can be present in each of the four main eating behaviour phenotypes. Group 1 low satiation, Group 2 low satiety, Group 3 emotional eating, Group 4 mixed phenotype.

For example, an individual in obesity phenotype Group 1 can have an eating behaviour score in the category of reduced satiation that is equal or greater than 1 standard deviations (1SD) than the mean of the predetermined reference sample for that eating behaviour. For example, an obesity phenotype Group 2 can have an eating behaviour score in the category of reduced satiety (constant cravers) that is equal or greater than 1 standard deviations (1SD) than the mean of the predetermined reference sample for that eating behaviour (reduced satiety). For example, an obesity phenotype Group 3 can have an eating behaviour score in the category of emotional eating that is equal or greater than 1 standard deviations (1SD) than the mean of the predetermined reference sample for that eating behaviour (emotional eating). For example, an obesity phenotype Group 4 can have several eating behaviours that have a similar score in two more categories, including reduced satiation, reduced satiety or emotional eating. These individuals have no single eating behaviour score that is equal or greater than 1 standard deviations (1SD) than any other eating behaviour score. Individuals in this category cannot characterised with the methods described herein.

The invention will now be illustrated with an application.

For example, once an eating behaviour phenotype has been identified, said human can be treated with a pharmacological intervention, surgical intervention, diet intervention, or behaviour intervention, and a treatment option can be selected for the individual after clinical examination and exclusion of contraindications to the treatment as suggested by the assessment algorithm (FIGS. 2, 3, 4 and 9). In some instances, identifying the eating behaviour phenotype requires the identification of one or more additional clinical variables and one or more additional assessments that are not included in the methods described here.

The following are examples of pharmacological interventions based on phenotypes of eating behaviour:

For example, when an individual is identified as having a low satiety phenotype (phenotype 1), based at least in part on the results of the assessment algorithm described herein, an appetite suppressant may be administered or the individual may be instructed to self-administer, including, but not limited to, such pharmacological agents as: phentermine, the combination phentermine-topiramate or other appetite suppressant (currently not FDA or EU approved for weight loss) such as lisdexamphetamine, or setmelanotide.

For example, when an individual is identified as having a low satiation phenotype (phenotype 2), based, at least in part, on the results of the assessment algorithm described herein, a glucagon like peptide one (GLP1) receptor analogue such as exenatide, lixisenatide, liraglutide, dulaglutide, semaglutide or dual GLP1 and GIP receptor analogues such as tirzepatide, or any similar peptides hormones acting on the GLP1 and the GIP receptor (currently in development) can be administered or the individual can be instructed to self-administer such a pharmacological agent.

For example, when an individual is identified as having an emotional eating phenotype (phenotype 3), based, at least in part, on the results of the assessment algorithm described herein, a combination of naltrexone-bupropion or an antiepileptic such as topiramate, or (currently not FDA or EU approved for weight loss) the combination of bupropion and zonisamide, tesofensine, or buspirone, or the individual can be instructed to self-administer such pharmacological agents.

(4) No dominant eating behaviour phenotype identified; the current approach of clinicians' choice based on clinicians' preferences and contraindications to medications may be used, as the methods described here do not imply that one treatment approach is superior to another.

Individualised pharmacological interventions for the treatment of obesity based on eating behaviour phenotypes as described herein may also include a combination of more than one specific pharmacotherapy intervention in the event that the testing algorithm identifies two similarly dominant eating behaviours. The pharmacotherapies that are discussed here may, in certain circumstances, be given to an individual as part of a combination therapy together with one or more other therapies or substances that are utilised in the treatment of obesity. A combined therapy utilised to treat an obese human, for example, can include providing one or more of the pharmacotherapies disclosed herein as well as one or more obesity treatments such as weight-loss operations (e.g. gastric sleeve, RYGB or single anastomosis bypass). When one or more of the pharmacotherapies described herein are used in conjunction with one or more other agents or therapies used to treat obesity, the additional agents or therapies can be administered or performed concurrently or separately. A pharmacotherapy may consist of an obesity pharmacotherapy, including for example an appetite suppressant, anticonvulsant, antidepressant, or an opioid antagonist. A pharmacotherapy may also be a formulation with controlled release.

Further details of one or more embodiments of the invention are described below. Other features and advantages of the invention will be apparent from the description and drawings, and from the claims.

According to one embodiment of the invention there is provided methods of identifying distinct eating behaviour described herein, there is a computer algorithm for calculating the dominant eating behaviour phenotype of said individual. The computer algorithm entails a computer-readable program based on established proprietary algorithms which are part of the present disclosure. The computer-readable program comprises instructions that, when executed by a processor, cause the processor to identify predominant eating behaviours, non-dominant eating behaviours and relationships between different eating behaviours. As said, one feature of the computer algorithm is determining the individual's dominant eating behaviour phenotype. The proprietary algorithms are written as computer-readable program code containing instructions that, when carried out by a processor, lead the processor to recognise said different eating behaviours.

In accordance with other embodiments of the invention, system, apparatus and computer analysis are employed to execute the described methods. The apparatus to analyse eating behaviours comprises a testing module operative to administer the test and an instruction module operative to instruct the individual regarding the test rules. For example, the methods and systems detailed herein may include a data analysis structure operative to store responses and related data. A data transmission interface may also enable communication with a remote device or a data cloud via a network. In some embodiments, an additional analysis module may control the preceding operation.

The operative aspects of the present invention are presented in the drawings 1-4.

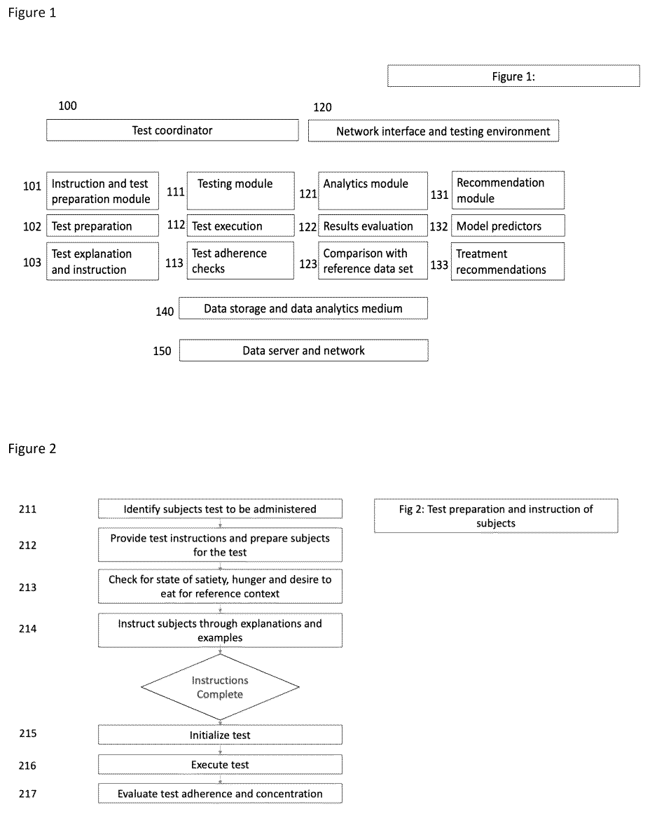

FIG. 1 is a simplified diagram illustrating components of the embodiment of the invention, detailing the four main components of the testing modules, including the instructions and test preparation module (101), the testing module (111), the test analytics module (121), and the clinical recommendations module (131).

The apparatus depicted in FIG. 1 may be embodied in a variety of devices and may incorporate all of the functionality and operational characteristics to analyse the results of the testing sequence and generate results as specified above for the differentiation of the different eating behaviour phenotypes. As indicated in FIG. 1, the embodiment may generally comprise an instruction module (101) used to prepare the individual taking the test (102). This instruction phase of the test may instruct the individual through a written set of instructions about the conduct of the test (103). In particular, the instruction module may include a test simulator, which may illustrate to the tested individual the structure of the test and appropriate interaction with the testing environment. Said instruction might improve, for example, test completion rates and reproducibility on subsequent test series.

The testing module (111) enables text execution and has inbuilt quality and adherence checks, documenting missing data and incomplete test completion. By way of example, the test items are presented to the individual taking the test in a random or pseudo-random order. The presentation of all text-based items is on a continuous scale (visual analogue scales), as detailed above. The image-based items include questions that are designed as a means to enhance and check attention and questions that are related to distinctive eating behaviours. Test execution (112) is done on a computer interface, which can be, for example, a desktop computer, a laptop computer, a tablet, a smartphone or any other respective interactive device that can perform the tasks described herein.

Test adherence checks (113) are designed to encourage the individual taking the test to complete all items with sustained attention and equal focus on the individual items. Different types of responses to the individual items provide the test trial information obtained during the testing phase. For example, a particular response to an item presented may include the selection of a specific position on a continuous scale by placing the virtual slider at that position. As noted above, the trial environment entails various items related to the different eating behaviour phenotypes. In some examples, the test is done by the individual tested in various clinical settings, as detailed above, including but not limited to self-administration of the test. For example, the testing time can be 25 to 30 minutes, depending on the proper responses required for the various items. The above-detailed modules are coordinated and controlled by a test coordinator module (100), ensuring all testing operations, including monitoring the configuration of the test and the progression of an individual through the test adhere to the test instructions. The analytics module (121) represents the data analytics structure and the computer algorithms to derive the individual eating behaviour scores of the individual tested, as detailed above. The test analysis may be performed by computer-executable instructions, program code or a combination thereof. For example, normative data related to the average test results for a particular reference population or control group will be stored in the data medium (140) to facilitate the calculation of the difference between the current individual result and the mean of the reference population.

Thereby, the reference database will grow with increasing numbers of individuals taking the test, improving the accuracy of the mean value for the reference population. The current individual test results and information derived therefrom may also be stored in the data medium (140). The testing software may operate with data records, historical data, and reference data sets and is likely maintained at said storage medium (140). Furthermore, the testing module (111) has access to the data storage medium (140), which may be embodied in a database, library file or other suitable data storage structure. All the functionality may be resident at a server or network (150), which in some embodiment can be a cloud service. In such an embodiment, for example, test data or results may be transmitted in whole or in part via a secure network. In some cases, the network interface may facilitate the communication between the testing software (111) and the analytics software (121) and enable communication with a remote server (150), as depicted in FIG. 1. The testing and analysis apparatus may also be implemented as an isolated system, hence, not connected to a network. Accordingly, the apparatus may be embodied in a computer workstation or desktop computer.

The clinical recommendation module (131) sets forth the information gathered during the test execution and the related data analysis in a presentable format to the individual tested and the respective clinician ordering and administering the test. In some examples, this can be an explanation in a written form providing detailed information about the test results, the meaning of the results for the individual, and any possible prospective recommended treatments as detailed above. In some examples, this can be depicted in graphs and charts illustrating the different eating behaviours and the relationship between them to the individual taking the test and the respective clinician ordering it. Any subsequent arrangements may incorporate all of the separate assessments set forth above.

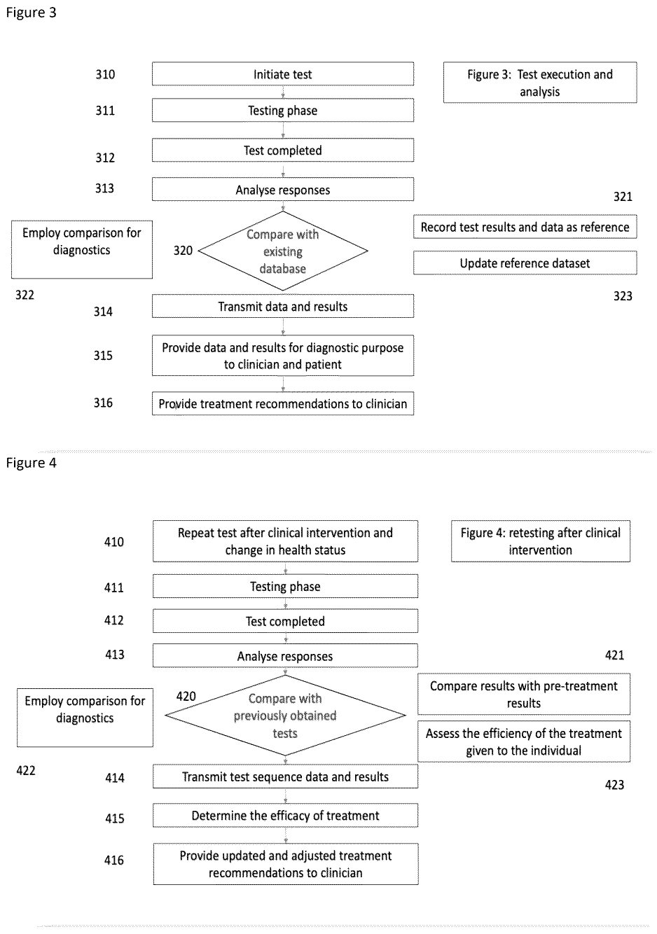

FIG. 2 is a simplified flow diagram illustrating the general operation of one embodiment of the testing method, facilitating the administration of a test sequence. The testing sequence has no set time limits, however, there may be general maximal time limits for administrative, logistical, or other reasons. Setting a time or clock mechanism for the test sequence might be appropriate. In reference to FIG. 2, blocks 212 through 217 represent an iterative approach to completing the steps in performing the test. The instruction phase for each individual taking the test corresponds to blocks 212 to 214 and has been described in more detail above. The testing phase corresponds to blocks 215 and 216 and is described in more detail in the paragraphs above. Upon completion of all items in the test sequence, as indicated in block 217, responses and results may be compiled as depicted in block 218. Individual test responses, data related to aspects of the responses, and information derived from both may be analysed as indicated in block 218 and further detailed in FIG. 3. Additionally, response data and information derived therefrom may be transmitted for analysis to a remote device, as depicted and described in FIG. 1. The extent to which trial data representative of the responses are analysed before transmission may depend on security and privacy concerns, as stipulated by local and respective jurisdictions and their regulatory framework.

FIG. 3 is a simplified flow diagram illustrating one embodiment of the process of deriving individual eating behaviour scores for each individual taking the test by comparing the individual scores, as set out below, with the summative data from the database generated during the evaluation and training phases of the algorithm and methods described herein. The test sequences, decision blocks 310 to 313, instructing and testing, compiling test results and analysing the test data and associated information may relate to the testing embodiment illustrated in FIG. 2 and described in detail above. In FIG. 3 embodiment, the test results may determine if an individual has a single eating behaviour that is more pronounced than other eating behaviours and how this eating behaviour sore compares to sores in the different eating behaviours, as illustrated in decision blocks 320 to 323. To determine whether an eating behaviour is dominant and henceforth suitable for a selective treatment with a pharmaceutical or else, a comparison of the individual's test results with a normative reference data set (block 320) will be made as set out above. In one embodiment of the method, the mean value of each eating behaviour score, including mean eating behaviour scores for group 1 (reduced satiety), group 2 (reduced satiation) and group 3 (emotional eating), are constantly refined by integrating ongoing testing results into the existing data set as depicted in decision blocks 221 and 223.

FIG. 4 is a simplified flow diagram illustrating one embodiment of the process of ascertaining the efficiency of a treatment regime. Decision blocks 411 to 417 correspond to the abovementioned descriptions, whereby treatment includes any form of pharmacological, surgical, dietary, or lifestyle intervention as set out above. As detailed in FIG. 4, the change in the test results over time, whereby the time interval can be any time between 3-18 months or even longer, indicates whether the treatment had an impact on the individual eating behaviour results by comparing the test results with previously obtained results with the same test in the same individual under identical test conditions. The treatment and re-testing periods may be customized to serve the needs of each individual taking the assessment. An individual taking the assessment may be referred herein as a subject. The test may be referred to as presenting questionnaire items and receiving responses.

In some embodiments, the repeat testing may be initiated more frequently than the interval noted above, for example, after metabolic surgery interventions or to assess the response to a particular pharmacological intervention where the selection based on EB characteristics was not entirely determined at the outset of treatment. Test results and any information obtained by comparing the responses before and after the clinical intervention and any associated intermediate, diagnostic or other related information may be transmitted to a remote server, for instance, for further analysis or storage as depicted in decision block 414. Where treatment has been given, and the efficacy of the treatment has been ascertained by repeating the test sequence, the responsible clinician may be informed to either continue, adjust or change the respective treatment, as depicted in block 416.

It is generally accepted in treating people with obesity that treatment can be considered effective or not after treatment for three months. Hence, repeating the testing after three months may represent the most suitable time interval to ascertain treatment efficacy. It is generally accepted that many factors impact the effectiveness of any treatment given, and the present disclosure is not intended to be limited by any clinical, empirical or context-specific factors that will affect the efficacy of any obesity treatment. An exemplary process of evaluating the effectiveness of a pharmaceutical is illustrated in FIG. 4; hence, the individual may return to block 410 after an appropriate interval (block 423).

It will be appreciated that several alterations or modifications may be implemented concerning the embodiments described above, and the order of the individual decision blocks is not intended to imply a specific sequence of operations to the exclusion of other possibilities. Any particular clinical situation may dictate the most efficient or appropriate sequence of operations, as given in FIGS. 2-4.

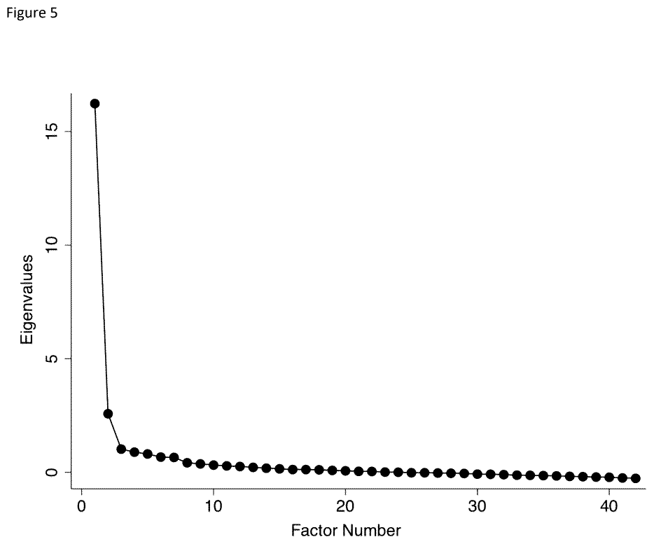

FIG. 5 shows aspects related to the explorative factor analysis, depicting the Eigenvalues of Initial Exploratory Factor Analysis Model. Factor Analysis was performed to determine the dimensionality of the 42-item tool. The initial Exploratory Factor Analysis retained 24 factors; however, only 3 had an eigenvalue [symbol] 1.0 Together, these 3 factors accounted for 85% of the observed variance. Based on these results and hypotheses that additional eating behaviour types such as binge eating and hedonistic eating may represent separate constructs, the selected models retained 3-7 factors. The models with 6 and 7 factors were eliminated because one or more of the factors did not have at least three items which loaded onto it. Models with 3-5 factors accounted for 85-92% of the variance observed. Stepwise removal of items with inadequate factor loading values resulted in one or more factors with fewer than three items on both the four- and five-factor models; therefore, a three-factor model was determined to be the best fit to the data. Further information about this process is provided in example one.

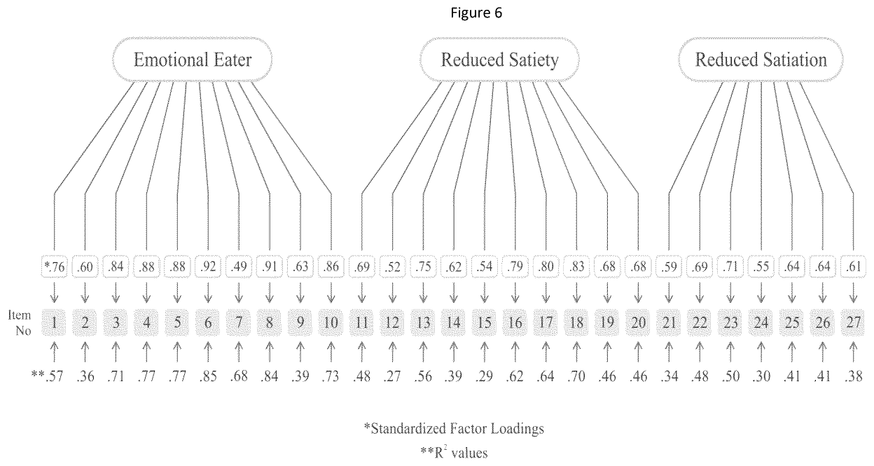

FIG. 6 is a simplified diagram and Table 1 a table depicting the Three-Factor Model of Eating Behaviour Type classification of the Confirmatory Factor Analysis (CFA), depicting *Standardized Factor Loadings and **R² values, factor analysis was performed to determine the fit of the model obtained through Exploratory Factor Analysis. All items loaded onto the expected factors as depicted in FIG. 6, and model fit statistics demonstrated a good model fit which explained 99.8% of the variance. Cronbach's internal consistency were similar to those obtained during Exploratory Factor Analysis: emotional eater ( [symbol] =0.95), feaster ( [symbol] =0.90), and constant craver α=0.82). Assessment of internal consistency by gender, ethnicity, age group, and diabetes diagnosis showed that all demographic sub-groups demonstrated adequate reliability. Further information about this process is provided in example one.

TABLE 1

| | Emotional Eating | | | | | | | | | | Reduced Satiety | | | | |
|---|---|---|---|---|---|---|---|---|---|---|---|---|---|---|---|
| * | 0.76 | 0.60 | 0.84 | 0.88 | 0.92 | 0.49 | 0.91 | 0.63 | 0.86 | 0.69 | 0.52 | 0.75 | 0.62 | 0.54 | 0.79 |
| ***. | 1 | 2 | 3 | 4 | 5 | 6 | 7 | 8 | 9 | 10 | 11 | 12 | 13 | 14 | 15 |
| ** | 0.57 | 0.36 | 0.71 | 0.77 | 0.77 | 0.85 | 0.68 | 0.84 | 0.39 | 0.73 | 0.48 | 0.27 | 0.56 | 0.39 | 0.29 |

| | Reduced Satiety | | | | Reduced Satiation | | | | | | | |
|---|---|---|---|---|---|---|---|---|---|---|---|---|
| * | 0.80 | 0.83 | 0.68 | 0.68 | 0.59 | 0.69 | 0.71 | 0.55 | 0.64 | 0.64 | 0.64 | 0.61 |
| ***. | 16 | 17 | 18 | 19 | 20 | 21 | 22 | 23 | 24 | 25 | 26 | 27 |
| ** | 0.62 | 0.64 | 0.70 | 0.46 | 0.46 | 0.34 | 0.48 | 0.50 | 0.30 | 0.41 | 0.41 | 0.38 |

* Standardised Factor Loadings
** R²
*** Item No.

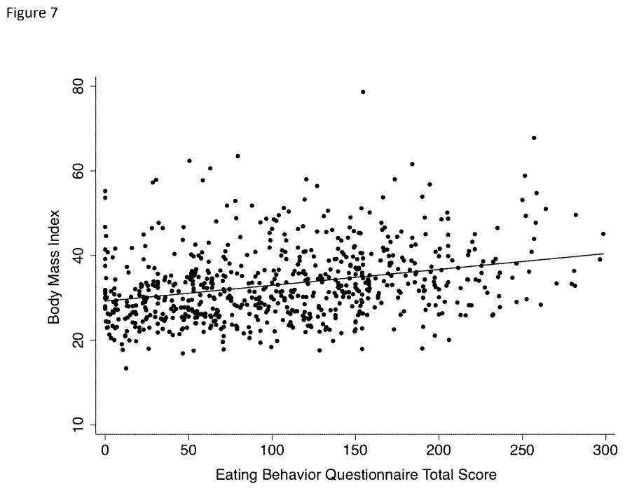

FIG. 7 depicts the associations between the aggregate eating behaviour scores (0-80) on the assessment tool and Body Mass Index (kg/m2). The aggregate Eating Behaviour score (the sum of three individual scores) was calculated to determine if a higher BMI was associate with stronger motivation to eat. As documented in FIG. 7, results showed a significant correlation (Spearman rho=0.314, P=0.0005) between the aggregate score and BMI (kg/m2).

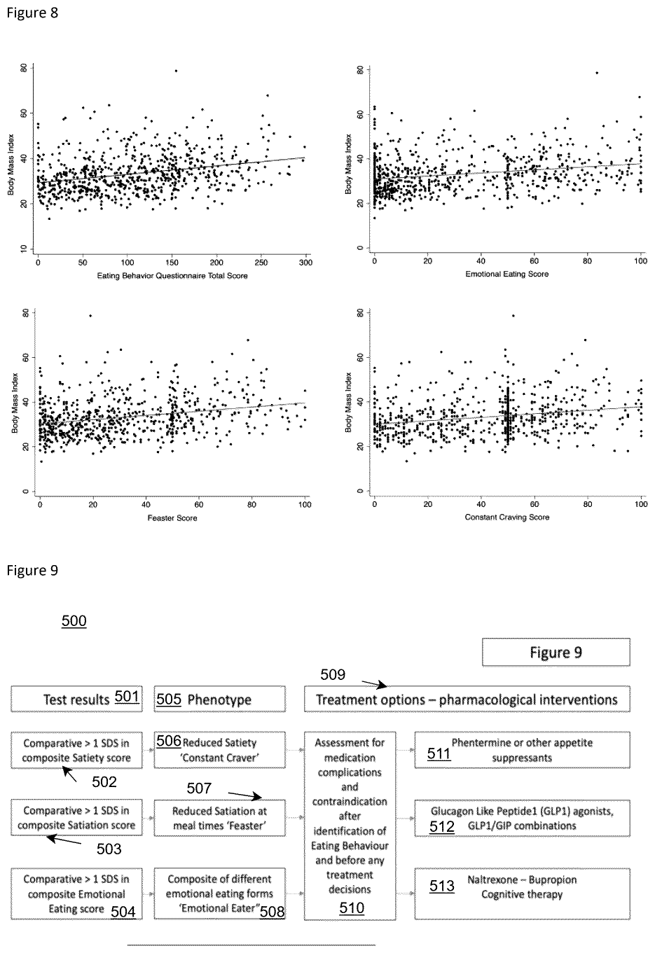

FIG. 8 (a-c) depicts the associations between the individual eating behaviour-specific scores and Body Mass Index (kg/m2). These scores were calculated to determine if any particular eating behaviour type correlated more strongly with BMI than others. These correlations were all significant but slightly weaker (Spearman rho=0.24 (emotional eater), 0.27 (feaster), and 0.25 (constant craver), p=0.0000), as shown in

FIG. 7 (b, c, d). This suggests that the system and methods of evaluating actionable eating behaviour phenotypes described herein can identify an obese human with a high body mass index (BMI) as having higher eating behaviour scores in the assessment tool.

FIG. 9 is a simplified flow diagram 500 illustrating exemplary treatment interventions for the eating behaviour groups identified, at least in part, based on eating behaviour phenotypes, as described in detail above.

FIG. 9 shows test result data 501. The results shown are comparative SD>1 in composite satiety score 502. Comparative Satiation score is shown as 503. Comparative>1SD in composite emotional eating score is shown as 504.

Phenotypes are shown as 505. Reduced satiety ‘Constant Craver’ is shown as 506. Reduced a=satiation at meal times is shown as 507. Composite of different emotional eating forms ‘Emotional eater’ is shown as 508. FIG. 9 depicts data associating test results with phenotypes: 502 with 506; 503 with 507, 504 with 508.

An assessment tool and step is depicted by 510. This tool assesses medical complications and counterindications after identification of Eating behaviour factor. FIG. 9 depicts with arrows data on the phenotypes being used by the tool or step 51.

FIG. 9 also depicts treatments or interventions that phenotypes are responsive to. In this case interventions 511 is Phentermine or other appetite suppressants. 512 depicts Glucagon Like Peptide (GLP-1) agonists, GLP1, GIP combinations. 513 depicts Naltrexone—Bupropion and Cognitive Therapy.

Validation of the testing algorithm will now be illustrated with a first example.

The invention will be further described in the following examples, which do not limit the scope of the invention described in the claims. The invention is based on the theoretical concept that it is possible to distinguish actionable obesity traits with a questionnaire-based clinical tool. The process of the invention was evaluated in a cohort of 908 individuals who consented and of whom 729 fully completed all assessment process steps. The enrolment was done after an initial pilot, a co-design phase with two participant focus groups, and feedback was integrated into the final tool. Participants reflected the Auckland, New Zealand/Aotearoa ethnicities (51% European, 12% Maori, 19.3% Pacifica and 15.5% Asian), a wide range of age groups (18-76 years), 57% male and 43% female, mainly type 2 diabetes (73%), with 68% being either overweight or obese and 76% wanting to lose weight. The appropriateness of factor analysis was assessed using Bartlett's test of sphericity and the Kaplan Meyer Olkin (KMO) measure of sampling adequacy. The final three-factor model retained 27 items (of 42) and accounted for 96% of the variance observed. The three factors identified correspond to the eating types: emotional eater (10 items), feaster (10 items), and constant craving (7 items).

Cronbach's test for internal consistency provided high estimates of reliability for all three scales: emotional eater (=0.94), feaster (=0.90), and constant craving (=0.83). Assessment of internal consistency by gender, ethnicity, and age group showed that all demographic sub-groups demonstrated adequate reliability (Cronbach alpha 0.89 to 0.96). Confirmatory factor analysis (CFA) was performed on the second half of the sample (n=364) to determine the fit of the model obtained through EFA. All items loaded onto the expected factors and model fit statistics demonstrated a good model fit which explained 99.8% of the variance. In a retest reliability cohort, asking a subset of participants to complete the same assessment tool a second time six weeks apart, the model showed moderate to good reliability (ICC values between 0.75-0.90). Using this strategy, 190 (26.1%) participants were classified as type E: emotional eaters, 162 (22.2%) as type F: feasters, and 322 (42.2%) as type C; and having constant cravings. Fifty-five (7.5%) participants could not be classified using the highest median score because they tied on two or more factors.

The invention described herein also entails image-based questions to improve the concentration of participants completing the questionnaire. These questions were designed de-novo and are based on the concept of vividness of imagery and mental imagery affecting food cravings. Such questions are generally more engaging, simple to answer, and can achieve sufficient quality of responses (Andrade, May, Deeprose, Baugh, & Ganis, 2014; Tiggemann & Kemps, 2005). In order to derive reliable answers it is important to provide images with familiarity, arousal and varying in valence (Seo, Rockers, & Kim, 2023). The initial has image-based questions with multiple-choice response format aiming to replicate the questionnaire's three-factor structure, allowing answers related to hunger, vividness of sensations or emotional eating. Attention enhancer items were included with the intention to ensure better participant engagement to assess every single question with persistent attention throughout the process of completing the questionnaire.

Sample size calculations were based on prior publications (Comrey & Lee, 1992) (Costello & Osborne, 2005) suggesting that a ratio of five participants for each independent variable is sufficient for reliable validation. Descriptive statistics were calculated for the demographics, weight and weight loss characteristics, and self-assessed EB type of participants. Responses that were included in analysis were compared to those which were excluded using Pearson chi-squared test and independent sample t-tests. The study sample was randomly divided and exploratory factor analysis (EFA) was performed on half the sample to determine the dimensionality of the NZ-EBQ. Confirmatory factor analysis (CFA) to confirm the findings from the EFA was performed using the second half of the sample (Knekta, Runyon, & Eddy, 2019). The appropriateness of factor analysis was assessed using Bartlett's test of sphericity and the Kaplan Meyer Olkin (KMO) measure of sampling adequacy. The normality of survey items was assessed and determined to be non-normal. Therefore, ranked variables were created for each item and used in factor analysis; Spearman coefficients were used instead of the standard Pearson coefficients. A principal factors process using orthogonal varimax rotation was used. In CFA, comparative fit index (CFI), Tucker-Lewis index (TLI), standardized root-mean-square residual (SRMR), and root-mean-square error of approximation (RMSEA) were calculated in order to assess the fit of the model (Comrey & Lee, 1992). Cronbach's alpha (α) was used to evaluate internal consistency of each factor in both EFA and CFA samples, as well as by demographic characteristics among the overall sample. Test-retest reliability of certain demographic characteristics were assessed using Kappa coefficient, where a value of 0.61-0.80 is ‘good’ and 0.81-1.00 is ‘very good’ (Landis & Koch, 1977). For the items included in the final construct model, intraclass correlation coefficients (ICC) were calculated to assess the consistency of measurement by participants at different times. A two-way mixed effects model requiring absolute agreement was used, where values between 0.50 and 0.75 indicate moderate reliability, 0.76-0.90 indicate good reliability, and values greater than 0.90 indicate excellent reliability (Koo & Li, 2016). Finally, participants were classified into one of three key EB types emotional eaters (EE), constant cravers (CC) describing reduced satiety, or feasters (F), describing reduced satiation, based on the highest median score observed among the factors. First, the median score of all items on each scale, or factor, were calculated. Then, these were compared and the highest of the three scores was considered the participant's primary EB type. For example, individuals who had a higher median score on the emotional eating factor than on the feaster or constant craving factor were classified as an emotional eater. Some participants could not be classified using the highest median score because they tied on two or more factors; these participants were excluded from further analyses. Median scores and interquartile range are reported for the overall sample as well as by EB classification. Kappa coefficient assessed the test-retest reliability of this classification. All data analysis was performed using Stata Statistical Software (Release 15, College Station, StataCorp, USA). A p value of <0.05 was considered significant for all statistical tests.

Exploratory Factor Analysis: The Phase I study sample (n=729) was randomly divided in order to perform exploratory factor analysis (EFA) and confirmatory factor analysis (CFA). Bartlett's test of sphericity indicated that there was sufficient correlation among items to run factor analysis (χ²=9491.21, p=0.000) and the Kaplan Meyer Olkin (KMO) measure of sampling adequacy suggested that half the total sample size was sufficient (KMO=0.95). Thus, EFA was performed on half the sample (n=365) to determine the dimensionality of the 42-item EB survey. The remaining half (n=364) were reserved for CFA to confirm the findings from the EFA (Knekta et al., 2019) This approach yielded a ratio of participants to items of >8:1, exceeding the minimum standard of 5:1.

Exploratory Factor Analysis—Round 1: The initial EFA retained 24 factors; however, only 3 had an eigenvalue [symbol] 1.0 Together, these 3 factors accounted for 85% of the observed variance. Based on these results and hypotheses that additional EB types such as binge eating and hedonistic eating may represent separate constructs (Gormally, Black, Daston, & Rardin, 1982; Stickney & Miltenberger, 1999) than the three previously identified in the literature such as satiety, satiation and emotional eating (Acosta et al., 2015; Camilleri & Acosta, 2016), EFA models retaining 3-7 factors were run. The models with 6 and 7 factors were eliminated because one or more of the factors did not have at least three items which loaded onto it. Models with 3-5 factors accounted for 85-92% of the variance observed. Because all three possible solutions (i.e., 3, 4, and 5 factor models) included several items with factor loadings [symbol] 0.40 on all factors on all factors, stepwise removal of these items was performed during a second round of EFA (Watkins, M. W, 2018). The factor loading of the exploratory factor analysis is depicted in FIG. 5.

Exploratory Factor Analysis—Round 2: Stepwise removal of items with inadequate factor loading values resulted in one or more factors with fewer than three items on both the four- and five-factor models; therefore, a three-factor model was determined to be the best fit to the data. To achieve factor loadings [symbol] .0.40 for all items in the three-factor model, nine items (21%) were eliminated using stepwise removal. At this point, there were multiple items that demonstrated cross-loading (i.e., factor loadings [symbol] .0.40 on multiple factors). Because cross-loading can distort factors by artificially inflating relationships (Howard, 2016, p. 59), stepwise analysis and removal of items that demonstrated cross-loading was conducted.

Specifically, any items that cross-loaded but met the 40-30-20 rule were retained (Howard, 2016). This rule states that items with a primary loading value of ≥0.40, a secondary loading value of 0.30, and a difference in loading values of ≥0.20 may be retained. Remaining items that demonstrated cross-loading were removed stepwise, beginning with those with a uniqueness of 0.50 and those with the lowest total factor loading values. The final three-factor model shown in Table 2 retained 27 items and accounted for 96% of the variance observed. The three factors identified correspond to the eating types presented in previous studies: emotional eater (10 items), feaster/reduced satiation (10 items), and constant craver/reduced satiety (7 items). Cronbach's test for internal consistency provided high estimates of reliability for all three scales: emotional eater (α=0.94), feaster (α=0.90), and constant craving (α=0.83).

Confirmatory factor analysis (CFA) was performed on the second half of the sample (n=364) to determine the fit of the model obtained through EFA. All items loaded onto the expected factors. FIG. 6 is a graphical representation of the Confirmatory Factor Analysis (CFA) of the Three-Factor Model of Eating. Model statistics demonstrated a good model fit which explained 99.8% of the variance. Specifically, both CFI and TLI values exceeded 0.90 and SRMR and RMSEA values were below 0.8. Cronbach's internal consistency in the half of the sample used for CFA were similar to those obtained during EFA: emotional eater (α=0.95), feaster (α=0.90), and constant craver (α=0.82).

Internal Consistency and Test-Retest Reliability: Assessment of internal consistency by gender, ethnicity, age group, and diabetes diagnosis showed that all demographic sub-groups demonstrated adequate reliability. Analysis of the matched responses from Phase I and II data collection showed that most demographic and weight loss characteristics also demonstrated very good reliability (Kappa coefficients≥0.81), which was performed as an indication of data quality, although self-assessed EB displayed only moderate reliability (0.43) (Appendix, Table A-3). Using ICC values, most of the 27 survey items included in the final construct model displayed moderate test-retest reliability (values between 0.50-0.75) while four items (5, 6, 8, and 10) showed moderate to good reliability (values between 0.75-0.90). Overall, the emotional eater and feaster scales demonstrated moderate to good reliability and the constant craving scale showed moderate reliability (Appendix, Table A-4).

Classifying Eating Behaviours of Participants: Participants were assigned to one of three EB types based on the highest median score among the three factors. For example, individuals who had a higher median score on the emotional eating factor than on the feaster or constant craving factor were classified as an emotional eater. Using this strategy, 190 (26.1%) Phase I participants were classified as emotional eaters, 162 (22.2%) as feasters, and 322 (44.2%) as having constant cravings. Fifty-five (7.5%) participants could not be classified using the highest median score because they tied on two or more factors. Generally, participants who were classified as emotional eaters had higher median scores on all factors although the factor measuring constant craving had the highest overall median score (49.00, IQR: 20.00-56.00)

Test-Retest Reliability of Classification: The same classification strategy was used to determine the EB type of participants in Phase 11 and then compared to Phase I in order to evaluate the impact of the moderate reliability of individual items. A Kappa coefficient of 0.46 (65.7% agreement) indicated moderate reliability for this model (Table 4). Participants classified as emotional eaters and those with constant cravings had a higher proportion of agreement (71.2% and 68.9%, respectively) than feasters (52.9%).

Relationship between Eating Behaviour Type and BMI: Even though we could assign a dominant EB trait to a substantial number of participants based on the highest median score among the three factors, all participants displayed multiple EBs. We calculated an aggregate EB score (the sum of three EB scores) to determine if a higher BMI was associated with stronger motivation to eat. We found a significant correlation (Spearman rho=0.314, P=0.0005) between the aggregate EB score and BMI. This association in depicted in FIG. 7. Eating behaviour-specific scores were also assessed to determine if any particular EB type correlated more strongly with BMI than others. These correlations were significant but slightly weaker (Spearman rho=0.24 (emotional eater), 0.27 (feaster), and 0.25 (constant craver), p=0.0000). The correlations between the individual eating behaviour scores and BMI are depicted in FIG. 8 (a, b, c, d), indicating that individuals with a higher BMI may generally have a stronger motivation to eat.

Validation of the testing algorithm in a separate cohort will now be illustrated as a second example.

The testing algorithm the invention was evaluation in a further cohort of 81 participants who provided consent and could fully complete the assessment. Confirmatory factor analysis yielded a good model fit where all items loaded onto the expected factors. Cronbach's a for each sub-scale (i.e., eating behavior type) exceeded the minimum accepted value of 0.70 and were similar to those observed in the first cohort: emotional eater (α=0.94), feaster (α=0.92), and constant craving (α=0.83).

Classifying Eating Behaviours of Participants: Participants were assigned to one of three eating behaviour types based on the highest median score of the factors. For example, individuals who had a higher median score on the emotional eating factor than on the feaster or constant craving factor were classified as an emotional eater. Using this strategy, 40.7% participants were classified as emotional eaters, 21.0% as feasters, and 28.4% as having constant cravings. Only 9.9% of participants could not be classified using the highest median score because they tied on two or more factors. Generally, participants who were classified as emotional eaters or feasters had much higher median scores for the associated factor compared to the other two factors; however, constant cravers yielded similar values across all three factors. Repeated analysis in a separate cohort with no participants with diabetes and a different demographic background confirmed the factor loading and the results obtained in the confirmatory factor analysis in example 1.

Treatment based on eating behaviour phenotypes will now be illustrated with a third example.

Treating patients with their eating behaviour congruent medication achieves significantly more weight loss than incongruent medication selection. This study is currently enrolling participants and anticipated completion date is 2^{nd} quarter 2024.

Study Design: In a 26 week, prospective, block randomised, double-blinded, single centre proof of concept study, participants with obesity are treated with their eating behaviour congruent medication (intervention) vs. incongruent medication (control), assessing for any differences in weight loss achieved.

Primary Objectives: To assess whether identifying predominant eating behaviour phenotypes, will improve pharmacotherapy selection in order to maximise weight loss and improvement in obesity related complications.

Secondary Objectives: To assess possible additional benefits of weight loss achieved with EB congruent medication allocation vs. EB non-congruent allocation vs. placebo. This will include glycaemic control (as assessed by change in HbA1c over six months), changes in Liver function test (ALT, AST and GGT), lipid profile (TC, TG, HDL-C, LDL-C), urate level, changes in serum creatinine and urinary albumin/creatinine ratio.

Study population: People aged 18-75 years with and without type 2 diabetes with a BMI range of 30-55 kg/m2 who are interested in losing weight with the help of weight management medications. Sample size assessment and power calculations identified a minimum of 67 participants in each cohort to detect a 10% difference in weight loss achieved using a two-sided test (at a level of 0.05) with approximately 80% power. Based on these calculations, we will recruit 75 participants into each group.

Eating behaviour congruent treatment group means that we match the medication to the participant's dominant eating behaviour trait, eating behaviour in-congruent group means that participants are treated with a medication that does not match their dominant eating behaviour. Since people can have more than one dominant eating behaviour score, for this proof-of-concept study we only select those with one non-dominant EB score, where the dominant EB score is at least one standard deviation greater than any other EB score. Hence, in order to be enrolled, the score on non-primary eating behaviour scales needs to be at least one standard deviations (of the mean primary eating behaviour score) lower than score of primary eating behaviour.

The results of the assessment tool are blinded to the participants and the investigator, and the computerised assignment is supervised by an independent observer (statistician), who is responsible for assigning participants to the intervention and control groups. Due to the different types of medications used (tablets vs. injections) it is not possible to blind participants to the medication they receive. To overcome this, we use randomisation based on blinding of congruence of medication type to eating behaviours (test results), hence response to one medication (e.g. Naltrexone/Bupropion combination) given at the same dose and treatment schedule, will be compared between EB congruent vs. EB in-congruent treatment groups. Hence, participants will know that they receive e.g. Naltrexone/Bupropion combination, but they will not know whether this medication is matching their eating behaviour phenotype. Participants from the three eating behaviour cohorts, as identified by said assessment process described herein, will be group randomised to either congruent or incongruent treatment at the time of enrolment.

Participants will now be described to further illustrate the invention.

A study cohort including men and women 18-75 years of age at the time of enrolment who have obesity, defined as WHO class 3 and 4 (Body mass Index 30-55 kg/m2).

Inclusion criteria will now be described to further illustrate the invention.

Benefiting and interested in medical weight loss as assessed by their clinician and the participant themselves.

Identified as having one dominant eating behaviour domain at the Auckland Eating behaviour questionnaire as detailed above:
People with and without type 2 diabetes;
Exclusion criteria;
People having received bariatric surgery;
People with type 1 diabetes;
People prescribed insulin of a Glucagon Like Peptide 1 (GLP1) agonist;
People on another weight loss medication;
People being diagnosed with a significant mental health condition;
People being prescribed medication that could interfere with any of the medications used in the study;
People diagnosed with or currently assessed for eating disorders;
Medical conditions or complications that would prevent the use of any of the three weight management medication;
People undergoing alcohol, opioid or benzodiazepine withdrawals;
Pregnancy, breast feeding, planning to become pregnant in the next year;
Study visits will now be described to further illustrate the invention.

Randomisation is done by an independent observer (statistician) based on a computer-generated algorithm. The statistician assigns participants to their respective medication (congruent or non-congruent) and informs the investigators of the treatment group allocations. Anthropometric measures (height, weight, waist circumference, blood pressure and pulse), blood and urine tests are obtained, and medical history is checked for possible exclusion criteria or contraindications to any of the study medications. The investigator discusses possible side effects of the selected medication and the medication titration algorithm, organises medication prescription and dispensing.

All participants are offered two diet counselling group sessions via a video conferencing tool, conducted by a registered dietitian, each lasting approximately 60 minutes. Participants have free access to an online diet and meal planning tool and the researchers access the app analytics at the end of the trial. Once participants have been on stable treatment for three month, they attend a follow up visit, during which repeated measurements for weight, waist circumference, blood pressure and pulse, blood tests, and safety checks are obtained. Participants are asked about any changes to their health, and repeat the assessment tool while on the medication to assess for any change to eating behaviours while on medication.

The study is completed after six months on the stipulated dose or the maximally tolerated dose. Participants are asked to repeat all measurements and blood tests. They are also asked to repeat the assessment tool once again to assess if any further change to eating behaviours are noted. Participants complete a semi-structured interview about their experiences during the study and changes in eating behaviour.

Analysis of co-primary outcomes will now be described to further illustrate the invention.

(1) The difference in total body weight loss achieved from baseline to 3 and 6 months after starting treatment, compared between the eating behaviour congruent vs. eating behaviour in-congruent medication allocation groups.

(2) The percentage of responders defined as number of participants losing 5% or more of total body, and 10% or more of total body weight at 3 and 6 months from baseline, compared between the eating behaviour congruent vs. eating behaviour in-congruent medication allocation groups.

Analysis of secondary outcomes will now be described.

To assess possible additional benefits of weight loss achieved with EB congruent medication allocation vs. EB non-congruent allocation vs. placebo. This will include glycaemic control (change in HbA1c over six months), changes in Liver function test (ALT, AST and GGT), lipid profile (TC, TG, HDL-C, LDL-C), and urate level. Renal outcome will be assessed by changes in serum creatinine and urinary ACR ratio.

To assess the adherence to either of the three study medications.

To assess side effects resulting in discontinuation or dose adjustment of study medication

To assess whether responses to the assessment toll will change after treatment with either of the study medications at the 3 and 6 months assessment visits.

Statistical analysis will take a number of forms depending on the data being considered. The core statistical tools are likely to be regression analysis and independent t-tests.

An interim analysis based on the observed coefficient of variation in the responses is conducted after 50% of the of the allocated participants have completed the study to ensure that the study has sufficient power based on the proposed study sample size.

### Other Embodiments Will Now be Described

While the invention has been described in conjunction with the detailed description thereof, the preceding description is intended to illustrate and not limit the scope of the invention, which is defined by the scope of the appended claims. Other aspects, advantages, and modifications are within the scope of the claims. Unless otherwise stated, all technical and scientific terms used herein have the same meaning as commonly understood in the art to which this invention pertains. Although methods similar or equivalent to those described herein can be used to practice the invention, suitable methods are described in this document. It will be understood that although several prior art publications are referred to herein, this reference does not constitute an admission that any of these documents form part of the common general knowledge of the field of endeavour in any country or that any reference constitutes prior art. In addition, the materials, methods, and examples are illustrative only and not intended to be limiting. Various changes and modifications to the presently preferred embodiments described herein will be apparent to those skilled in the art. Such changes and modifications may be made without departing from the present disclosure's spirit and scope and diminishing its attendant advantages.

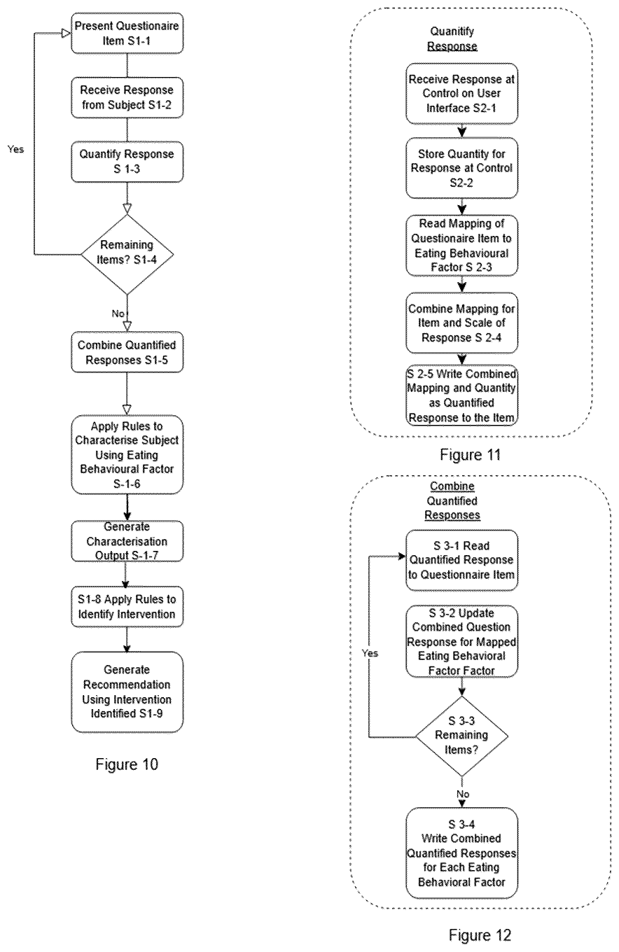

FIG. 10 shows a process according to an embodiment of the present invention. In this particular embodiment the process is a process to allow a subject to be classified as having an eating behaviour phenotype. This process may refer to a test as used above.

At Step S 1-1 a questionnaire item is presented to a subject to be classified at a user interface of a computer terminal. The questionnaire item of this particular embodiments is a question. The question item may be an ‘item’ as used above.

At Step S 1-2 the user interface receives a control input at the user interface to allow the subject to respond to the questionnaire item.

At Step S 1-3 the control input is quantified. In this particular embodiment the user interface allows the subject to input a response that is quantified as between 1 and 5. At S 1-3 also a quantity is stored for the response to the questionnaire item.

At step S 1-4 the process determines whether there are remaining questionnaire items in a set of questionnaire items that will be used to characterise the subject. If there are remaining questions the process returns S-1 to presenting another questionnaire item. If the complete questionnaire items of a complete set of questionnaire items has been presented the process moves to step S-5.

At S 1-5 the process combines the quantified values stored for each questionnaire item at S-3. This provides a combined quantified value for questionnaire responses which is stored.

At Step S 1-6 the process characterises the subject using the combined quantified value. In this particular embodiment the process applies rules to the combined values.

At step S 1-7 the process generates and stores data which carries information on the characterisation made at step S-7.

At step S 1-8 the process determines an intervention dependent on the characterisation data generated at S-7. The characterisation data is read and analysed to determine a medical intervention. In this embodiment the intervention is determined using stored rules. In one example, an intervention known to be efficacious for treating a phenotype identified in the characterisation data from step S-6.

At step S 1-8 the process generates data carrying information on the intervention determined at step S-7. In this embodiment the data carries information on a recommendation for a clinician to consider.

FIG. 11 shows a process of quantifying responses such as performed at S-3 according to an embodiment of the present invention.

At step S 2-1 responses to questionnaire items are received at a control provided on a user interface operated by the subject. The user interface may be remote from a central system implementing the process of FIG. 2.

At step S 2-2 data carrying information on a quantity or value for the response at S-2-1 is generated and stored. This provides a quantitative response to the respective questionnaire item. This may be referred to herein as a scale of response to a questionnaire item.

At step S 2-3 the process reads a mapping of the respective questionnaire item to an eating behavioural factor.

At step S 2-4 the process generates and stores data carrying information on a combining of the quantitative value stored at S-2-2 and the mapping to behavioural factor of S-2-3. In one example, a quantitative value of 0.8 on a scale of 0 to 1 may be stored at step S-2-2. This may indicate a strong reaction, such as strong agreement with the questionnaire item. In this example the mapping value for the given questionnaire item that this response was made to may have a mapping of 0.6 to the eating behavioural factor referred to as Emotional Eating (EE). In this particular embodiment the process combines the quantified response and mapping using a scalar multiplication, such as 0.8×0.6 to get 0.42.

At step S 2-5 the process stores the combined value as a combined quantified-mapped value for a given response by the subject.

The reader will recognise that the behavioural factors here may be selected by exploratory factor analysis and/or confirmatory factor analysis illustrated above. The reader may recognise these factors as the Eigenfunctions illustrated above. The combined quantified-mapped value and mapping to a behavioural factor illustrated with reference to FIG. 10 may be recognised by the reader as the Eigenvalues illustrated above. The reader will recognise that the subject can be characterised using these Eigenfunctions or behavioural factors. Here a characterisation is a description using defined characteristics, such as behavioural factors.

The reader will recognise the set of questionnaire items of the process of FIG. 10 as the result of stepwise elimination of items illustrated above. In one example the questionnaire items are the result of elimination of questionnaire items from a superset of questionnaire items. In one example questionnaire items that are not eliminated, or which may be described as selected from the superset, are those that sufficiently map to a behavioural factor that the set of behavioural factors can be used to characterise a subject. The reader will further recognise that if, the selected set of questionnaire items each approximately exclusively map to a behavioural factor, the behavioural factors needed to characterise subjects can be minimised. For example, a set of factors can account for a defined variance observed in a sample of subjects. The reader will recognise the selected questionnaire items as providing a good model fit where all items/questionnaire items loaded onto the expected factors as confirmed by Confirmatory Factor Analysis illustrated above. Here loaded onto is analogous to mapping.

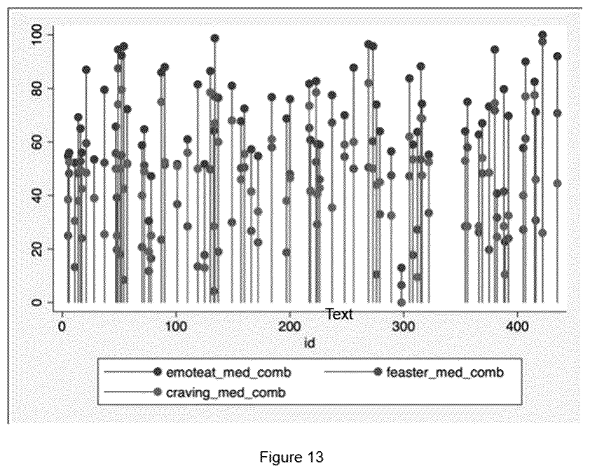

FIG. 12 shows a process of combining quantified responses and mapping values such as performed at step S 2-4.

At step S 3-1 a combined quantitative value for a response to a questionnaire item, that has been stored at Step S-2-2, is read.

At step S-3 data for the response to all questionnaire items presented so far is updated with the quantitative response read at step S 3-1.

If it is determined at S3-3 that the last questionnaire item in a set has been read, the process moves to Step S 3-4.

At step S 3-4 the process combines the combined quantitative eating responses for each eating behavioural factor. For example, the set of combined values found using each quantitative response to each questionnaire item and the mapping value the respective each questionnaire items may be combined over the factor Emotional Eating (EE). The same step of combining is performed for each eating behavioural factor.

At step S 3-3 the process S 3 determines whether there are remaining questionnaire items in the set of questionnaire items. If there are, the process iterates by returning to S3-1 to read another quantitative response.

In another step not depicted data carrying information on the scale of responses is stored at a repository for use factor analysis. This provides an iteratively growing repository of data to use in developing factor models, which will be recognised as selecting factors for revised processes.

In another embodiment process of generating data carrying information on a treatment recommendation for a clinician, the process comprising the steps of:
presenting a set of questions to a subject at a computer implemented user interface, each question having a defined loading of one of a set of behavioural factors;
receiving responses to the set of questions from the subject at the computer implemented user interface;
quantifying each response to represent a scale of the response;
combining the response with a mapping of the item to its respective behavioural factor to generate data carrying information representing the scale of response and mapping of the response to the behavioural factor;
combining the quantified responses for each behavioural factor to generate data carrying information on combined responses for each behavioural factor in the set;
applying a computer implemented analytical process to characterise the subject using the behavioural factors to generate data carrying information on a characterisation of the subject by behavioural factor.

The process may comprise generating data carrying information which identifies a treatment selected from a set of treatments, the selection made using the characterisation of the subject by behavioural factors.

The process may comprise generating data carrying information which identifies a treatment by identifying a phenotype using the behavioural factor.

The process may generating data carrying information which identifies a treatment using data which associates a behavioural factor with a phenotype to identify a phenotype for the subject.

The process may comprise generating data carrying a selected recommendation from a set of candidate recommendations of a intervention, the recommendation selected using the characterisation.

The process may comprise generating data carrying a selected recommendation using data which associates a set of phenotypes with a set of treatments known to be effective in treating the phenotype.

The process may comprise generating data carrying information which recommends the selected intervention.

In other steps not process may comprise:
repeating the steps of the paragraph above in a second iteration of the process of the paragraph above to provide first and second iterations of the process;
generating data carrying information to record a change in the characterised behaviour factors in the subject; and
generating data carrying information on efficacy of the intervention recommended in the first iteration.

The process stores the data carrying information on responses by scale of response and/or mapping of response to provide updated data to use in selecting behavioural factors.

In another step not depicted data carrying information on the scale of responses combined with the mapping of responses is stored at a repository for use factor analysis.

In another step not depicted data carrying information on the scale of responses combined with the mapping of responses is stored at a repository for use factor analysis and combined over items for each respective factor that a subset of items map to above a threshold, or which map sufficiently exclusively to.

The reader will appreciate that alternative embodiments map present items that map to a wider set of factors while the characterisation and/or categorisation of the subject uses only the selected factors or the model with reduced number factors to characterise the subject. The reader will appreciate this may be advantageous to collecting data for use in factor analysis and/or selecting factors or configuring a factor model for revised process. The reader will appreciate that this may have the disadvantage of presenting the subject with more questions.

FIG. 13 shows a set of quantitative responses combined with mapping values for responses of a subject to a set of questionnaire items. In this example three eating behavioural factors are depicted: emotional eating (EE), feasting (FE) or low satiety, and Constant Craving (CE) or low satiation. Each vertical line depicts a response to a questionnaire item by the subject combined with a mapping value for the respective questionnaire item to a behavioural factor. In this example a user interface (not shown) receives a control input from the subject and quantifies the response and a quantitative value determined for the response is combined with a predetermined value which indicates how the specific questionnaire items maps to a an eating behavioural factor. In one example the user interface receives a control input that allows the subject user to indicate a degree or response to the questionnaire item. In one specific example the user interface presents a question and the subject as user inputs a response indicating the degree to which they relate to the question. The mapping of that particular questionnaire item to eating behavioural factors is read and multiplied with the quantitative value for the subject's response to that questionnaire item. Each vertical line shown in FIG. 4 depicts a quantitative response with a mapping value, which will be referred to herein as mapped-response value or mapped-response data point.

The prior art items and items developed by the applicants, as illustrated above, provide a superset of items that are known to indicate given eating behaviour factors. For example, an item: “When you are going through a stressful or upsetting setting time, what happens to your eating?” may be known to indicate the factor Emotional Eating.

In some embodiments assigning participants or subjects to one of three eating behaviour types based on the highest median score of the factors, to classify them is an example of characterising the subject by eating behaviour factors. In various embodiments the highest median score is a combined quantitative-mapped value. In some examples this is generated at S 2-4. In other examples alternative analysis to the highest median is used.

The characterisation of a subject, such as depicted by step S-1-6, is performed using the quantitative-mapped values for the complete set of questionnaire items. Each mapped-response value, shown as a vertical plot line in FIG. 4, represents a response to a different questionnaire item presented to a subject. The collective set of mapped-response value is used by embodiments of the present invention to characterise the subject. In this embodiment the characterisation is by behaviour factor. In one example the set of mapped-response values is used to identify an eating behavioural factor that is dominant over the other eating behavioural actors in the set of eating behaviour factors. The factors in a given iteration of the process, being the factors that a subject may be characterised with, are represented by the questionnaire items selected to be used in the iteration of the process.

In some embodiments a phenotype is known as being responsive to a one of a set of treatments. In these embodiments, a process as illustrated with reference to FIGS. 1 and 2 will be a process of identifying an obese subject as being responsive to one of a given set of treatments.

In alternative embodiments to that illustrated with reference to FIG. 10, the characterisation of a subject may apply alternative methods to rules. In one embodiment machine learning may be applied to the combined quantified values for responses to questionnaire items. In one embodiment machine learning may be applied to the quantified values stored at the step illustrated with reference to S-3 for responses to questionnaire items. In this example the quantified responses may not have been combined.

In alternative embodiments a step equivalent to S 1-6 may use alternative analysis to rules. In one additional embodiment the characterisation may be by heuristics. In one additional embodiment the characterisation may be by a machine learning facility. In various embodiments this may be a machine learning engine. In various embodiments this may be a machine learning model. In further alternative embodiments the characterisation performed at a step equivalent to S-6 may be performed by applying data analytics.

In further embodiments the recommendation data generated in a step equivalent to S 1-8 is carried in a transmission to a central server and/or to remotely located devices to transmit a recommendation to a physician operating the device.

In alternative embodiments the combining at a step equivalent to S 2-4 may use an alternative to scalar multiplication. In one alternative embodiment the step may multiply a vector by a scalar. In a further alternative embodiment, the process may combine the quantified response with the mapping value as an inner product between two vectors. In a further alternative embodiment, the process may combine the quantified response with the mapping value as an inner product between matrices representing the quantified response value and mapping.

In alternative embodiments of the invention the process, such as illustrated with reference to FIGS. 1 to 3, may combine the quantitative responses to questionnaire items with mapping values at a later stage than illustrated. In one alternative embodiment the quantitative responses to questionnaire items and the mapping values may be combined at a step similar to S3-2. The reader will appreciate that the various embodiments including the embodiments illustrated with reference to FIG. 3 and the alternative embodiment mentioned here all find a combined value for each eating behaviour factor to the responses of a subject to questionnaire items while taking into account how each questionnaire item maps to an eating behavioural factor. This will provide a combined value for each eating behavioural factor. These combined values over the set of eating behavioural values is analysed according to various embodiments to characterise the subject making the responses to questionnaire items. In some embodiments the characterisation is a categorisation, where possible, into categories defined by eating behaviour factor. In some examples analysis performed by embodiments identifies that no single category can be identified.

In some embodiments the categorisation made by analysis identified a dominant eating behavioural factor. In these embodiments further analysis exemplified by reference to step S1-8 of FIG. 1 identifies a medical intervention and/or treatment that is likely to be efficacious for a given behavioural factor. The applicant has made a non-trivial break-through that eating behavioural factors as can be identified from factor analysis correlate to phenotypes. In some embodiments the factor analysis identifies eating behavioural factors by factor analysis of a sample of responses by subjects to a set of questionnaire items which are known to be relevant to a medical condition. In these embodiments further analysis exemplified by reference to step S1-8 of FIG. 1 identifies a medical intervention and/or treatment that is likely to be efficacious for a given defined eating behavioural factor.

In some embodiments the characterisation is a categorisation, where possible, into categories defined by phenotype. In these embodiments further analysis exemplified by reference to step S1-8 of FIG. 1 identifies a medical intervention and/or treatment that is likely to be efficacious for a given phenotype.

In some embodiments the characterisation is a categorisation, where possible, into categories of subject where the categories are defined by likely efficacy of a given medical intervention.

In alternative embodiments a questionnaire item is a question with an image.

In one specific example the user interface presents a question and an image for the subject as user to input a response indicating the degree to which they relate to the question.

In another example a questionnaire item may be an image without necessarily being accompanied by a text question.

In one specific example a user enters a response to a questionnaire item by entering a number.

A quantitative value is generated by a process of embodiments of the invention reading the number. In one specific example a user enters a response to a questionnaire item by selecting a response that indicates a degree of agreeing, disagreeing or reacting in general to a questionnaire item. A quantitative value is generated by a process of embodiments of the invention reading a number associated with each selected response. In various alternative embodiments any known process of allowing a subject to input a quantifiable response to a questionnaire item may be used to generate a quantitative value for a response to a questionnaire item.

In alternative embodiments behavioural factors not necessarily relate to eating replaces eating behavioural factors in the claims and examples above.

Embodiments of the present invention described herein enable a questionnaire-based process to assess and determine distinct eating behaviours, which may be responsible for the varied responses to available weight management medications.

Embodiments of the present invention described herein enable computer implemented process to assess and determine distinct eating behaviours, which may be responsible for the varied responses to available weight management medications.

Various embodiments of the present invention present a significant advantage in assessing eating behaviour phenotypes compared with current or otherwise proposed assessment tools. In some embodiments, the methods described herein may be used in conjunction with other methods of assessing or diagnosing distinct eating behaviours. For example, it has been described that the gastric emptying time of solids and liquids can be used to describe individuals with reduced satiation at meal times and rapid return to hunger. Other tests to assess reduced satiety are also available, such as assessments of stomach capacity, postprandial levels of satiation hormones, and calorie intake in a buffet-style meal. Various embodiments of the present invention may be used in conjunction with one or more of the above test, but not limited to the test described herein.

In particular, the methods described herein permit testing in multiple clinical settings, such as self-assessment of individuals, assessment in a primary care clinical setting, or assessment prior to a dedicated medical specialist assessment. In certain circumstances, the assessment can be completed at the time of a clinical assessment, and in others, it can be completed prior to the assessment. Additionally, the present invention can be administered more rapidly in terms of study time than was previously feasible.

For example, if the assessment might be used by the investigator or clinician to design and direct individualised treatment options. The procedures described here allows for simple assessment repetition over time. For example, after the initiation of a treatment, such as the administration of a drug for weight loss, a dietary intervention, or a surgical procedure, allowing for surveillance of treatment responses to the respective treatment approaches. For example, the methods described herein can be utilised to anticipate future weight loss responsiveness, to minimise weight loss plateaus, and to improve weight loss maintenance in some circumstances.

For example, when treating an individual with obesity, that individual may have one or multiple co-morbidities related to obesity. In some instances, the described methods and materials can be used to treat obesity-related co-morbidities. Type 2 diabetes, dyslipidaemia, hypertension, obstructive sleep apnoea, gastroesophageal reflux disease, arthritis in weight-bearing joints, metabolic associated liver disease (MAFLD), and atherosclerosis (coronary artery disease and/or cerebrovascular disease) are examples of obesity-related co-morbidities. When treating obesity as described in the present disclosure, for example, the treatment can be effective in reducing weight, waist circumference, haemoglobin A1c, or fasting glucose or any clinical measures used to assess the severity or progression of any of the above obesity associated co-morbidities. Individuals examined using the methods described herein can be of most ethnicities as the tool was validated in a cohort of people of several ethnicities (Caucasians, Pacific Islanders, Maori, and Asians). However, the instrument has not yet been evaluated in Africans and African-Americans.

Various embodiments of the invention provide a process that allows a subject to be classified as having an eating behaviour phenotype responsive to an intervention based upon the response to questionnaire items used in the process.

Various embodiments of the invention provide a questionnaire-based process which is able to indicate one or more eating behavioural factors applicant taking advantage of the applicant's observation that by selecting questionnaire items that map to a selected set of eating behavioural factors and which can approximate or be assumed orthogonal, a questionnaire-based process can indicate one or more eating behavioural factors. The applicant has further observed that by assuming eating behavioural factors correlate to phenotypes and/or underlying physiology, a questionnaire-based process can indicate a phenotype and an intervention with known efficacy for the phenotypes and/or underlying physiology.

Embodiments of the invention may allow an eating behaviour phenotype of a subject to be identified by a questionnaire-based process.

A process of embodiments may allow questionnaire-based discovery of physiological phenomena.

A process of embodiments of the invention may allow an eating behaviour phenotype to be indicated using an assumption that eating behaviour factors correlate to eating behaviour phenotypes.

In various embodiments and aspects of the invention a loading onto a factor is a mapping and the terms are interchangeable.

In various additional embodiments actionable eating behaviour characteristics are eating behavioural traits.

In various additional embodiments actionable eating behavioural traits are assumed to correlate to eating behavioural phenotypes.

In various additional embodiments actionable eating behaviour characteristics are eating behavioural phenotypes.

In alternative embodiments the systems and processes mentioned herein are implemented by a computer and processes. In these embodiments steps and outputs are performed and provided as data generated, written and read carry information described by the systems and processes mentioned. For example, ‘administering a test’ comprises presenting data carrying information on items at a user interface of computer system and/or receiving inputs from the test subject at the computer interface and/or preforming computer operations on data corresponding to the steps described herein. For example also, ‘operative to diagnose eating behaviours' comprises generating and/or storing data carrying information indicating a eating behaviour trait and/or eating behaviour phenotype. Also for example, ‘identifying an intervention’ comprises generating and/or storing data carrying information which identifies an intervention’. Also for example, ‘providing recommendations for an intervention’ comprises generating and/or storing data carrying information which provides recommendations for an intervention. Also for example, ‘instruct a subject’ comprises generating and/or storing data carrying information to instruct a subject. For example also, ‘recording responses to test questions’ comprises generating and/or storing data carrying information to record responses to test questions. Also for example ‘guiding treatment’ comprises generating and/or storing and/or displaying data carrying information to guide treatment. Further computer and data implementations of steps of processes and systems described herein will be apparent to the reader.

In some embodiments stepwise elimination of items or questionnaire items is a process by which selecting items from a superset of items. In alternative embodiments selection of items is performed by including items into a set of selected items. For example including items in a selected set may be based on low cross-loadings.

In some embodiments selection of a model defined by factors is a process by which factors are selected.

In various alternative embodiments additional or alternative factors may be use. For example 4 factors may be used. For example 5 factors may be used.

In various models items and/or factors are selected to satisfy a defined criteria with data for tests for a population or sample of respondents. For example the criteria may be a defined variance.

In various additional embodiments factor analysis may be used to select a set of factors by reference to sample data. In some embodiments the factors may be selected by a defined number of items that load onto the factors. In some embodiments factors with 4 or less items loading onto the factor may be eliminated. In some embodiments factors with 4 or less items loading onto the factor may be eliminated. In some embodiments factors with 10 or less items loading onto the factor may be eliminated. In some embodiments factors with 100 or less items loading onto the factor may be eliminated. In some embodiments factors with at least a defined number of items loading onto the factor may be selected into a set of factors.

In various additional embodiments factors may be selected such that a model representing the factors accounts for a minimum specified variance in a sample or population. In some embodiments selection of factors may be performed so as to minimise the number of factors that account for a defined variance. In various additional embodiments factors may be selected such that a model representing the factors accounts for a minimum specified variance in a sample or population. The defined variance in some additional embodiments may be 70% or more. The defined variance in some additional embodiments may be 80% or more. The defined variance in some additional embodiments may be 85% or more. The defined variance in some additional embodiments may be 90% or more.

In various additional embodiments factors may be selected such that one or more specific factor related to a trait and/or phenotype of interest is selected and other factors are selected to minimise the set of factors including said selected one or more factors that satisfy a defined criteria for a sample or population of subjects r participants.

Various alternative embodiments factors and/or traits and/or phenotypes of conditions alternative to eating are used. These factors, traits or phenotypes may relate to Genetic syndromes. These factors, traits or phenotypes of alternative embodiments may relate to any conditions known by the reader.

In further embodiments the items or questionnaire items are presented and responses received at a computer generated user interface which is remote from a computer module performing analysis.

In further embodiments there are telehealth computer system operable to perform any process as described and illustrated herein. The system provides an advantage that a subject can be tested remotely from a clinician and without any need for blood tests.

In further embodiments there is a system for treating a human or mammal subject comprising: a data store with data carrying information to present as a set of questionnaire items to a subject. In this example, this is a data store which stores the questionnaire items.

The system of this embodiments data store with data carrying information defining a mapping of each questionnaire item to a respective behaviour factor of a behavioural factor model having a set of behaviour factors. For example, the data store holds the data shown in FIG. 6. Here each questionnaire item has a mapping value describing how it maps to a given factor. This value is used by combining it with a quantitative value for a response by a subject to an item. For example, an item would be displayed, a response with a scale, say 0 to 100, is received from the subject and the mapping and scale, or a normalised scale is multiplied to get a combined-mapped response. A response may be 90, which is normalised to 0.9 and multiplied by the mapping factor 0.76 to the factor emotional eating for item 1 in FIG. 6.

The embodiment has a testing processor operable generate data for a user interface to present the set of questionnaire items to a subject and receive control inputs from the subject and generate data carrying information quantifying each response. In one example the questionnaire items have been selected to each have a mapping value of 0.4 or more to one factor and less that 0.2 to any other. The factor that the item maps 0.4 may be the respective factor for the item, because items with cross-mapping to more than one factor have not been selected for the set of items for the test of the present example. The combined mapping values, E.g. 0.9×0.76, for the respective items, E.g. item 1 of FIG. 6, for each factor, E.g. Emotional Eating, Reduced Satiety and Reduced Satiation are analyses, E.G. by rules to identify a dominant factor or by machine learning applied to the complete set of combined mapped quantitative responses. The analytical system of this particular embodiment identifies a dominant behavioural factor.

A data store associating factors to treatments is read to for data associating behavioural factors with treatments to identify a treatment dependent on the output of the analytical tool. In one embodiment the data store associating factors identified in the test or processes illustrated herein being associated with phenotypes are depicted by arrows between 502 and 506, 503 and 507, and between 504 and 508. The analytical tool is not depicted but generates the characterisations depicted as 502, 503 and 504. In this embodiment data associating phenotypes 506, 507, and 508 with treatments 511, 512 and 513 are depicted by arrows from 506, 507 and 507 to 510 and arrows from 510 to 511, 512 and 513.

The multiple behavioural factors may be selected dependent on a set of data from responses of a sample of subjects. The multiple behavioural factors may be selected dependent on a set of data from responses of a sample of subjects combined with mappings of questionnaire items with respective factors. The factors may be selected dependent on the variance of the subjects responses to a set of questionnaire items.

Identifying characteristics that reliably distinguish people with obesity to enhance response rates to different obesity treatments has proven challenging. Previous disclosures have tried to detect distinguishable and actionable obesity phenotypes, yet most obesity management is empirical and still based on clinical judgment and individual factors. A simple tool as an additional part of the clinical assessment process that helps classify patients based on their dominant eating behaviour phenotype before commencing obesity treatment could help select the most appropriate therapies. The current disclosure supports the factors of reduced satiety, reduced satiation, and emotional eating as distinct eating behaviour phenotypes. The disclosure proposes identifying individuals with dominant eating behaviour and treating them with their eating behaviour congruent intervention, which may improve outcomes in the abovementioned measures. Previous studies have used existing questionnaires in addition to clinical investigations, e.g. gut hormone and metabolome signatures, or gastric emptying time of solids, tested by scintigraphy using 99mTc-colloid or validated nutrient drink satiation tests to measure satiation and postprandial fullness in relation to drinking a liquid nutrient or other related art to identify obesity phenotypes. However, such methods have limited use in routine clinical use where a time-efficient point-of-care tool will have additional benefits, as detailed above. The system and process described herein are easy to use in regular clinical care, allowing the clinician to choose the most suitable obesity medication for that individual, and by doing so, the response in weight loss and improvement of obesity-related complications may be greater than choosing medications based on clinician's choice, i.e. based on contraindications and preferences of patient and clinician. The process described herein provides instant feedback to the user about their eating behaviour, and this can be used in subsequent counselling and education sessions, hence increasing awareness of their eating behaviours and helping with additional therapies related to particular eating behaviours, whose descriptions are outside of this disclosure.

### REFERENCES

Acosta, A., Camilleri, M., Abu Dayyeh, B., Calderon, G., Gonzalez, D., McRae, A., . . . Clark, M. M. (2021). Selection of antiobesity medications based on phenotypes enhances weight loss: A pragmatic trial in an obesity clinic. Obesity (Silver Spring, Md.), 29(4), 662-671. doi:10.1002/oby.23120 [doi]
Acosta, A., Camilleri, M., Shin, A., Vazquez-Roque, M. I., Iturrino, J., Burton, D., . . . Zinsmeister, A. R. (2015). Quantitative gastrointestinal and psychological traits associated with obesity and response to weight-loss therapy. Gastroenterology, 148(3), 537-546.e4. doi:S0016-5085(14)01423-1 [pii]
Aitken, R. C. (1969). Measurement of feelings using visual analogue scales. Proceedings of the Royal Society of Medicine, 62(10), 989-993. doi:10.1177/003591576906201005
Andermann, M. L., & Lowell, B. B. (2017). Toward a wiring diagram understanding of appetite control. Neuron, 95(4), 757-778. doi:S0896-6273(17)30512-3 [pii]
Andrade, J., May, J., Deeprose, C., Baugh, S. J., & Ganis, G. (2014). Assessing vividness of mental imagery: The Plymouth sensory imagery questionnaire. British Journal of Psychology (London, England: 1953), 105(4), 547-563. doi:10.1111/bjop.12050 [doi]
Apovian, C. M., Aronne, L. J., Bessesen, D. H., McDonnell, M. E., Murad, M. H., Pagotto, U., . . . Endocrine Society. (2015). Pharmacological management of obesity: An endocrine society clinical practice guideline. The Journal of Clinical Endocrinology and Metabolism, 100(2), 342-362. doi:10.1210/jc.2014-3415 [doi]
Camilleri, M. (2019). Gastrointestinal hormones and regulation of gastric emptying. Current Opinion in Endocrinology, Diabetes, and Obesity, 26(1), 3-10. doi:10.1097/MED.0000000000000448 [doi]
Camilleri, M., & Acosta, A. (2016). Gastrointestinal traits: Individualizing therapy for obesity with drugs and devices. Gastrointestinal Endoscopy, 83(1), 48-56. doi:S0016-5107(15)02741-8 [pii]
Comrey, A. L., & Lee, H. B. (1992). A first course in factor analysis (2. ed. ed.). Hillsdale, NJ, Erlbaum.
Costello, A., & Osborne, J. (2005). Best practices in exploratory factor analysis: Four recommendations for getting the most from your analysis. Practical Assessment, Research & Evaluation, 10, 7. Retrieved from https://search.proquest.com/docview/2366831151
Couper, M. P., Tourangeau, R., Conrad, F. G., & Singer, E. (2006). Evaluating the effectiveness of visual analog scales. Social Science Computer Review, 24(2), 227-245. doi:10.1177/0894439305281503
Gibbons, C., Hopkins, M., Beaulieu, K., Oustric, P., & Blundell, J. E. (2019). Issues in measuring and interpreting human appetite (satiety/satiation) and its contribution to obesity. Current Obesity Reports, 8(2), 77-87. doi:10.1007/s13679-019-00340-6 [doi]
Gormally, J., Black, S., Daston, S., & Rardin, D. (1982). The assessment of binge eating severity among obese persons. Addictive Behaviors, 7(1), 47-55. doi:10.1016/0306-4603(82)90024-7
Gummer, T., RoRmann, J., & Silber, H. (2021). Using instructed response items as attention checks in web surveys: Properties and implementation. Sociological Methods & Research, 50(1), 238-264. doi:10.1177/0049124118769083
Härdle, W., & Simar, L. (2015). Applied multivariate statistical analysis (Fourth Edition ed.). Berlin; Heidelberg: Springer.
Howard, M. C. (2016). A review of exploratory factor analysis decisions and overview of current practices: What we are doing and how can we improve? International Journal of Human-Computer Interaction, 32(1), 51-62. doi:10.1080/10447318.2015.1087664
Knekta, E., Runyon, C., & Eddy, S. (2019). One size doesn't fit all: Using factor analysis to gather validity evidence when using surveys in your research. CBE Life Sciences Education, 18(1), rm1. doi:10.1187/cbe.18-04-0064
Koo, T. K., & Li, M. Y. (2016). A guideline of selecting and reporting intraclass correlation coefficients for reliability research. Journal of Chiropractic Medicine, 15(2), 155-163. doi:10.1016/j.jcm.2016.02.012
Landis, J. R., & Koch, G. G. (1977). The measurement of observer agreement for categorical data.
Biometrics, 33(1), 159-174. doi:10.2307/2529310
MacLean, P. S., Rothman, A. J., Nicastro, H. L., Czajkowski, S. M., Agurs-Collins, T., Rice, E. L., . . .
Loria, C. M. (2018). The accumulating data to optimally predict obesity treatment (ADOPT) core measures project: Rationale and approach. Obesity (Silver Spring, Md.), 26(Suppl 2), S6-S15. doi:10.1002/oby.22154
Roberts, C. A., Christiansen, P., & Halford, J. C. G. (2017). Tailoring pharmacotherapy to specific eating behaviours in obesity: Can recommendations for personalised therapy be made from the current data? Acta Diabetologica, 54(8), 715-725. doi:10.1007/s00592-017-0994-x [doi]
Ryan, D. H., & Yockey, S. R. (2017). Weight loss and improvement in comorbidity: Differences at 5%, 10%, 15%, and over. Current Obesity Reports, 6(2), 187-194. doi:10.1007/s13679-017-0262-y [doi]

1. A process of generating data carrying information on a treatment recommendation for a clinician, the process comprising the steps of:
presenting a set of questions to a subject at a computer implemented user interface, each question having a defined loading of one of a set of behavioural factors;
receiving responses to the set of questions from the subject at the computer implemented user interface;
quantifying each response to represent a scale of the response;
combining the response with a mapping of the item to its respective behavioural factor to generate data carrying information representing the scale of response and mapping of the response to the behavioural factor;
combining the quantified responses for each behavioural factor to generate data carrying information on combined responses for each behavioural factor in the set; and
applying a computer implemented analytical process to characterise the subject using the behavioural factors to generate data carrying information on a characterisation of the subject by behavioural factor.

2. The process of claim 1 comprising generating data carrying information which identifies a treatment selected from a set of treatments, the selection made using the characterisation of the subject by behavioural factors.

3. The process of claim 1 comprising generating data carrying a selected recommendation from a set of candidate recommendations of an intervention, the recommendation selected using the characterisation.

4. The process of claim 1 comprising repeating the steps of the paragraph above in a second iteration of the process of the paragraph above to provide first and second iterations of the process;
generating data carrying information to record a change in the characterised behaviour factors in the subject; and
generating data carrying information on efficacy of the intervention recommended in the first iteration.

5. The process of claim 1 comprising storing the data carrying information on responses by scale of response and/or mapping of response to provide updated data to use in selecting behavioural factors.

6. A process of treating a human subject comprising treating the subject with the identified treatment of claim 2.

7. The process of claim 1, further comprising storing the data carrying information on characterisations to provide updated data for an expanded sample of a population to use in selecting behavioural factors.

8. The process of claim 1 wherein said indication is defined as a value of a mapping metric for the item mapping to the eating behavioural factor.

9. The process of claim 1 wherein said describing may be defined as a value of a mapping metric for the item mapping to the eating behavioural factor.

10. The process of claim 1 comprising quantifying the received responses.

11. The process of claim 1 wherein characterising the subject comprises categorising the subject by eating behavioural factor.

12. The process of claim 1 wherein characterizing the subject comprises applying rules which categorise the subject by a dominant eating behavioural factor.

13. The process of claim 1 wherein characterising comprises applying analysis which identifies the highest value for a factor for the subject and which reference to two background variables:
(1) the median value (or score) for that eating behaviour (EB) in a whole reference data population (>1SD of the mean); either or
(2) the difference between the individual's dominant vs. second or third eating behaviour score. (>20% difference primary vs next).

14. A process of guiding treatment of obesity in a human, wherein said process comprises:
administering a test operative to diagnose eating behaviours in a said human;
identifying an intervention based on said eating behaviour characteristic obtained from said human by administering said test operative to diagnose eating behaviours;
instructing a human regarding rules for said test; and
providing recommendations for an intervention to said human to guide treatment.

15. The process of claim 14, wherein said test is administered to a human.

16. The process of claim 14, wherein said instructing comprises providing rules for performing said test.

17. The process of claim 14, wherein said administering said test comprise utilizing a computerised system and analytics algorithm.

18. The process of claim 14 further comprising analysing said responses and said data relative to previously recorded data sets.

19. The process of claim 14 wherein said previously recorded data records are obtained during previous administration of the said test.

20. The process of claim 14 wherein said previously recorded data records are normative data for a population previously administered said test.

21. The process of claim 18 wherein said analysing comprises identifying eating behaviour characteristics of said human.

22. A system comprising:
a testing module operative to administer a test; and
an instruction module operative to instruct a subject regarding rules for said test.
